SEQUENCE LISTING (49 sequences)

SEQ ID NO: 1 - DNA, 30 bp, Artificial sequence
  PCR-Primer
aaaggatccatcctcaccccggaacaagtt

SEQ ID NO: 2 - DNA, 30 bp, Artificial sequence
  PCR-Primer
aaaggatcccgatatgctttgccaacggac

SEQ ID NO: 3 - DNA, 30 bp, Artificial sequence
  PCR-Primer
aaatctagagtgagcaagggcgaggagctg

SEQ ID NO: 4 - DNA, 31 bp, Artificial sequence
  PCR-Primer
aaatctagattacttgtacagctcgtccatg

SEQ ID NO: 5 - DNA, 31 bp, Artificial sequence
  PCR-Primer
aaatctagaaccatgattacggattcactgg

SEQ ID NO: 6 - DNA, 33 bp, Artificial sequence
  PCR-Primer
aaatctagattatttttgacaccagaccaactg

SEQ ID NO: 7 - DNA, 30 bp, Artificial sequence
  PCR-Primer
aaaagtacttcaatgaacaagggcccaacc

SEQ ID NO: 8 - DNA, 29 bp, Artificial sequence
  PCR-Primer
aaaagtactgctcttcttgcgcagctggc

SEQ ID NO: 9 - DNA, 29 bp, Artificial sequence
  PCR-Primer
aaaggatcctctgccgacggggcagaggc

SEQ ID NO: 10 - DNA, 29 bp, Artificial sequence
  PCR-Primer
aaaggatcctttcttctcctccggggctt

SEQ ID NO: 11 - DNA, 31 bp, Artificial sequence
  PCR-Primer
aaaagtactagttttatgctcagatcgctgg

SEQ ID NO: 12 - DNA, 31 bp, Artificial sequence
  PCR-Primer
aaaagtactgcatccgttgcagttgatccac

SEQ ID NO: 13 - DNA, 29 bp, Artificial sequence
  PCR-Primer
aaaggatccgcggaccgcgaccgcagcgg

SEQ ID NO: 14 - DNA, 28 bp, Artificial sequence
  PCR-Primer
aaaggatcccgaggaagccctgccgccg

SEQ ID NO: 15 - DNA, 30 bp, Artificial sequence
  PCR-Primer
aaaggatccgcgctgacggtggcgacgctg

SEQ ID NO: 16 - DNA, 30 bp, Artificial sequence
  PCR-Primer
aaaggatcccgccgtgttggcgaggcacgt

SEQ ID NO: 17 - DNA, 1537 bp, Mycobacterium smegmatis
aagctttctagcagaaataattcattctgaacagaccccgccgtcgacacgaggagacacccaccatggccgccggacagcagcgccgccccaacctcctgctgccgttggtgcgtctgacccacctcgcggagtcggcgatcgaacgcgtgctcgcggactcgtcgctcaagatcgaggactggcgcgtgctcgacgagttggccggacggcgcaccgtgcccatgagcgatctcgcgcaggccacgctgatcacgggtccgactctcaccagaaccgtcgatcgccttgtgtcgcaagggatcatctaccggactgccgatctgcatgaccgccggcgggtgctcgtggcgttgaccccgcgggggcggacgctgcgcaaccgcctggtggacgcggtagccgaggccgagtgtgcggcttttgaatcgtgcgggctggacgtcgaccagttgcgcgaactcgtcgacaccacctcgaatttgacttcgtaaccacccgcgcccggccggcgttcacccttgacttttattttcatctggatatatttcgggtgaatggaaaggggtgaccatgccgacctacacattccgttgttcccactgcggtcccttcgatctcacctgcgcgatctccgagcgcgatgcggcggcgacctgtccggagtgccggacgccggcgcgccgggtcttcggttcggtagggctgacgacattcaccgcgggacatcaccgcgcattcgacgcggcgtccgcgagcgccgaaagtcccacggtggtgaagtcgattcccgcaggcgcggaccgcccgcgggccccgcgccgcaatcccggtctaccgagtctgccgaggtactagcgacatgggtggcgtcgggctcttctacgtgggtgcggtgctcatcatcgacgggctgatgctgctgggccgcatcagcccacgaggcgcaacaccgctgaacttcttcgtcggcggactgcaggtggtgacgcctacggtgctgatcctgcagtccggcggagacgcggccgtgatcttcgcggcctccgggctctacctgttcggcttcacctacctgtgggtggccatcaacaacgtgaccgactgggacggagaaggtctcggatggttctcgctgttcgtcgcgatcgccgcactcggctactcgtggcacgcgttcaccgccgaggccgacccggcgttcggggtgatctggctgctgtgggcagtgctgtggttcatgctgttcctgctgctcggcctggggcacgacgcactggggcccgccgtcgggttcgtcgcggtggccgaaggcgtgatcaccgccgccgtgccggccttcctgatcgtgtcgggcaactgggaaaccggcccgctccccgccgcggtcatcgccgtgatcggttttgccgcagttgttctcgcataccccatcgggcgccgtctcgcagcgccgtcagtcaccaaccctccaccggccgcgctcgcggccaccacccgataagagaaagggagtccacatgcccgaggtagtttt

SEQ ID NO: 18 - DNA, 579 bp, Ralstonia eutropha
  CDS (1)..(579)
atgatcctcaccccggaacaagttgcagcagcgcaaaaggccaacctcgaaacgctgttcggcctgaccaccaaggcgtttgaaggcgtcgaaaagctcgtcgagctgaacctgcaggtcgtcaagacttcgttcgcagaaggcgttgacaacgccaagaaggcgctgtcggccaaggacgcacaggaactgctggccatccaggccgcagccgtgcagccggttgccgaaaagaccctggcctacacccgccacctgtatgaaatcgcttcggaaacccagagcgagttcaccaaggtagccgaggctcaactggccgaaggctcgaagaacgtgcaagcgctggtcgagaacctcgccaagaacgccccggccggttcggaatcgaccgtggccatcgtgaagtcggcgatctccgctgccaacaacgcctacgagtcggtgcagaaggcgaccaagcaagcggtcgaaatcgctgaaaccaacttccaggctgcggctacggctgccaccaaggctgcccagcaagccagcgccacggcccgtacggccacggcaaagaagacgacggctgcctga

SEQ ID NO: 19 - protein, 192 aa, Ralstonia eutropha
MILTPEQVAAAQKANLETLFGLTTKAFEGVEKLVELNLQVVKTSFAEGVDNAKKALSAKDAQELLAIQAAAVQPVAEKTLAYTRHLYEIASETQSEFTKVAEAQLAEGSKNVQALVENLAKNAPAGSESTVAIVKSAISAANNAYESVQKATKQAVEIAETNFQAAATAATKAAQQASATARTATAKKTTAA

SEQ ID NO: 20 - DNA, 720 bp, Aequorea victoria
  CDS (1)..(720)
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 21 - protein, 239 aa, Aequorea victoria
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 22 - DNA, 1332 bp, Artificial sequence
  fusion protein
atgcccgaggtagttttcggatccatcctcaccccggaacaagttgcagcagcgcaaaaggccaacctcgaaacgctgttcggcctgaccaccaaggcgtttgaaggcgtcgaaaagctcgtcgagctgaacctgcaggtcgtcaagacttcgttcgcagaaggcgttgacaacgccaagaaggcgctgtcggccaaggacgcacaggaactgctggccatccaggccgcagccgtgcagccggttgccgaaaagaccctggcctacacccgccacctgtatgaaatcgcttcggaaacccagagcgagttcaccaaggtagccgaggctcaactggccgaaggctcgaagaacgtgcaagcgctggtcgagaacctcgccaagaacgccccggccggttcggaatcgaccgtggccatcgtgaagtcggcgatctccgctgccaacaacgcctacgagtcggtgcagaaggcgaccaagcaagcggtcgaaatcgctgaaaccaacttccaggctgcggctacggctgccaccaaggctgcccagcaagccagcgccacggcccgtacggccacggcaaagaagacgacggctgccggatccagtacttctagagtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 23 - protein, 443 aa, Artificial sequence
  Synthetic Construct
MPEVVFGSILTPEQVAAAQKANLETLFGLTTKAFEGVEKLVELNLQVVKTSFAEGVDNAKKALSAKDAQELLAIQAAAVQPVAEKTLAYTRHLYEIASETQSEFTKVAEAQLAEGSKNVQALVENLAKNAPAGSESTVAIVKSAISAANNAYESVQKATKQAVEIAETNFQAAATAATKAAQQASATARTATAKKTTAAGSSTSRVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 24 - DNA, 3687 bp, Artificial sequence
  fusion protein
atgcccgaggtagttttcggatccatcctcaccccggaacaagttgcagcagcgcaaaaggccaacctcgaaacgctgttcggcctgaccaccaaggcgtttgaaggcgtcgaaaagctcgtcgagctgaacctgcaggtcgtcaagacttcgttcgcagaaggcgttgacaacgccaagaaggcgctgtcggccaaggacgcacaggaactgctggccatccaggccgcagccgtgcagccggttgccgaaaagaccctggcctacacccgccacctgtatgaaatcgcttcggaaacccagagcgagttcaccaaggtagccgaggctcaactggccgaaggctcgaagaacgtgcaagcgctggtcgagaacctcgccaagaacgccccggccggttcggaatcgaccgtggccatcgtgaagtcggcgatctccgctgccaacaacgcctacgagtcggtgcagaaggcgaccaagcaagcggtcgaaatcgctgaaaccaacttccaggctgcggctacggctgccaccaaggctgcccagcaagccagcgccacggcccgtacggccacggcaaagaagacgacggctgccggatccagtacttctagaaccatgattacggattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgttggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaataa

SEQ ID NO: 25 - protein, 1228 aa, Artificial sequence
  Synthetic Construct
MPEVVFGSILTPEQVAAAQKANLETLFGLTTKAFEGVEKLVELNLQVVKTSFAEGVDNAKKALSAKDAQELLAIQAAAVQPVAEKTLAYTRHLYEIASETQSEFTKVAEAQLAEGSKNVQALVENLAKNAPAGSESTVAIVKSAISAANNAYESVQKATKQAVEIAETNFQAAATAATKAAQQASATARTATAKKTTAAGSSTSRTMITDSLAVVLQRRDWENPGVTQLNRLAAHPPFASWRNSEEARTDRPSQQLRSLNGEWRFAWFPAPEAVPESWLECDLPEADTVVVPSNWQMHGYDAPIYTNVTYPITVNPPFVPTENPTGCYSLTFNVDESWLQEGQTRIIFDGVNSAFHLWCNGRWVGYGQDSRLPSEFDLSAFLRAGENRLAVMVLRWSDGSYLEDQDMWRMSGIFRDVSLLHKPTTQISDFHVATRFNDDFSRAVLEAEVQMCGELRDYLRVTVSLWQGETQVASGTAPFGGEIIDERGGYADRVTLRLNVENPKLWSAEIPNLYRAVVELHTADGTLIEAEACDVGFREVRIENGLLLLNGKPLLIRGVNRHEHHPLHGQVMDEQTMVQDILLMKQNNFNAVRCSHYPNHPLWYTLCDRYGLYVVDEANIETHGMVPMNRLTDDPRWLPAMSERVTRMVQRDRNHPSVIIWSLGNESGHGANHDALYRWIKSVDPSRPVQYEGGGADTTATDIICPMYARVDEDQPFPAVPKWSIKKWLSLPGETRPLILCEYAHAMGNSLGGFAKYWQAFRQYPRLQGGFVWDWVDQSLIKYDENGNPWSAYGGDFGDTPNDRQFCMNGLVFADRTPHPALTEAKHQQQFFQFRLSGQTIEVTSEYLFRHSDNELLHWMVALDGKPLASGEVPLDVAPQGKQLIELPELPQPESAGQLWLTVRVVQPNATAWSEAGHISAWQQWRLAENLSVTLPAASHAIPHLTTSEMDFCIELGNKRWQFNRQSGFLSQMWIGDKKQLLTPLRDQFTRAPLDNDIGVSEATRIDPNAWVERWKAAGHYQAEAALLQCTADTLADAVLITTAHAWQHQGKTLFISRKTYRIDGSGQMAITVDVEVASDTPHPARIGLNCQLAQVAERVNWLGLGPQENYPDRLTAACFDRWDLPLSDMYTPYVFPSENGLRCGTRELNYGPHQWRGDFQFNISRYSQQQLMETSHRHLLHAEEGTWLNIDGFHMGIGGDDSWSPSVSAEFQLSAGRYHYQLVWCQK

SEQ ID NO: 26 - DNA, 1554 bp, Mus musculus
  CDS (1)..(1554)
atgtcaatgaacaagggcccaaccctgctggatggagacctccctgagcaggagaacgtgctccagagagttctgcagctgcctgtggtgagcgggacctgtgagtgcttccagaagacctacaacagcaccaaagaagcccaccccctggtggcctctgtgtgcaatgcctatgagaagggtgtacagggtgccagcaacctggctgcctggagcatggagccggtggtccgtcggctgtccacccagttcacagctgccaatgagttggcctgcagaggcctggaccacctggaggaaaagatcccggctcttcaataccctccagaaaagatcgcctctgaactgaagggcaccatctctacccgccttcgaagcgccaggaacagcatcagtgtgcccattgcaagcacctctgacaaggttctgggggccactctggccggctgcgagcttgccttggggatggccaaagagacagcagaatatgccgccaacacccgggttggccgactggcctctggaggggctgatctggctctgggaagcatcgagaaggtggtagagttcctcctgccaccagacaaggagtcagccccttcttccggacggcagaggacccagaaggctcccaaggccaaaccaagccttgtgaggagggtcagcaccctggccaacactctttctcgacacaccatgcaaaccacagcatgggccctgaagcagggccactctctggccatgtggatcccgggtgtggcacccctgagcagcctggcccagtggggcgcatcggcagccatgcaggtggtgtcccggcggcagagtgaggtgcgggtgccctggctgcacaacctggcagcctctcaggatgagagccatgacgaccagacagacacagagggagaggagacagacgacgaggaggaggaagaagagtccgaggctgacgagaacgtgctcagagaggttacagccctgcccaacccgagaggcctcctgggtggtgtggtacacaccgtgcagaacactctccggaacaccatctccgcagtgacctgggcacctgcggctgtgctgggcacggtgggaaggatcctgcacctcacaccagcccaggctgtctcctctaccaaagggagggccatgtccctatccgatgccctgaagggtgttacggataacgtggtagacactgtggtacactatgtgccgcttcccaggctgtccctgatggagcccgagagcgaattccgagacatcgataacccttcagcagaggcggagcgcaaagggtccggggcgcggcccgccagcccggagtccaccccgcgcccgggccagccccgcggcagcttgcgcagcgtgcggggtctcagcgcgccctcctgccccggcctggacgacaaaaccgaggcgtcagcgcgtcccggcttcctggctatgcccagagagaagcctgcgcgcagagtcagcgacagcttcttccggcccagcgtcatggagcccatcctgggccgcgcgcagtacagccagctgcgcaagaagagctga

SEQ ID NO: 27 - protein, 517 aa, Mus musculus
MSMNKGPTLLDGDLPEQENVLQRVLQLPVVSGTCECFQKTYNSTKEAHPLVASVCNAYEKGVQGASNLAAWSMEPVVRRLSTQFTAANELACRGLDHLEEKIPALQYPPEKIASELKGTISTRLRSARNSISVPIASTSDKVLGATLAGCELALGMAKETAEYAANTRVGRLASGGADLALGSIEKVVEFLLPPDKESAPSSGRQRTQKAPKAKPSLVRRVSTLANTLSRHTMQTTAWALKQGHSLAMWIPGVAPLSSLAQWGASAAMQVVSRRQSEVRVPWLHNLAASQDESHDDQTDTEGEETDDEEEEEESEADENVLREVTALPNPRGLLGGVVHTVQNTLRNTISAVTWAPAAVLGTVGRILHLTPAQAVSSTKGRAMSLSDALKGVTDNVVDTVVHYVPLPRLSLMEPESEFRDIDNPSAEAERKGSGARPASPESTPRPGQPRGSLRSVRGLSAPSCPGLDDKTEASARPGFLAMPREKPARRVSDSFFRPSVMEPILGRAQYSQLRKKS

SEQ ID NO: 28 - DNA, 2301 bp, Artificial sequence
  fusion protein
atgcccgaggtagttttcggatccagtacttcaatgaacaagggcccaaccctgctggatggagacctccctgagcaggagaacgtgctccagagagttctgcagctgcctgtggtgagcgggacctgtgagtgcttccagaagacctacaacagcaccaaagaagcccaccccctggtggcctctgtgtgcaatgcctatgagaagggtgtacagggtgccagcaacctggctgcctggagcatggagccggtggtccgtcggctgtccacccagttcacagctgccaatgagttggcctgcagaggcctggaccacctggaggaaaagatcccggctcttcaataccctccagaaaagatcgcctctgaactgaagggcaccatctctacccgccttcgaagcgccaggaacagcatcagtgtgcccattgcaagcacctctgacaaggttctgggggccactctggccggctgcgagcttgccttggggatggccaaagagacagcagaatatgccgccaacacccgggttggccgactggcctctggaggggctgatctggctctgggaagcatcgagaaggtggtagagttcctcctgccaccagacaaggagtcagccccttcttccggacggcagaggacccagaaggctcccaaggccaaaccaagccttgtgaggagggtcagcaccctggccaacactctttctcgacacaccatgcaaaccacagcatgggccctgaagcagggccactctctggccatgtggatcccgggtgtggcacccctgagcagcctggcccagtggggcgcatcggcagccatgcaggtggtgtcccggcggcagagtgaggtgcgggtgccctggctgcacaacctggcagcctctcaggatgagagccatgacgaccagacagacacagagggagaggagacagacgacgaggaggaggaagaagagtccgaggctgacgagaacgtgctcagagaggttacagccctgcccaacccgagaggcctcctgggtggtgtggtacacaccgtgcagaacactctccggaacaccatctccgcagtgacctgggcacctgcggctgtgctgggcacggtgggaaggatcctgcacctcacaccagcccaggctgtctcctctaccaaagggagggccatgtccctatccgatgccctgaagggtgttacggataacgtggtagacactgtggtacactatgtgccgcttcccaggctgtccctgatggagcccgagagcgaattccgagacatcgataacccttcagcagaggcggagcgcaaagggtccggggcgcggcccgccagcccggagtccaccccgcgcccgggccagccccgcggcagcttgcgcagcgtgcggggtctcagcgcgccctcctgccccggcctggacgacaaaaccgaggcgtcagcgcgtcccggcttcctggctatgcccagagagaagcctgcgcgcagagtcagcgacagcttcttccggcccagcgtcatggagcccatcctgggccgcgcgcagtacagccagctgcgcaagaagagcagtactgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 29 - protein, 766 aa, Artificial sequence
  Synthetic Construct
MPEVVFGSSTSMNKGPTLLDGDLPEQENVLQRVLQLPVVSGTCECFQKTYNSTKEAHPLVASVCNAYEKGVQGASNLAAWSMEPVVRRLSTQFTAANELACRGLDHLEEKIPALQYPPEKIASELKGTISTRLRSARNSISVPIASTSDKVLGATLAGCELALGMAKETAEYAANTRVGRLASGGADLALGSIEKVVEFLLPPDKESAPSSGRQRTQKAPKAKPSLVRRVSTLANTLSRHTMQTTAWALKQGHSLAMWIPGVAPLSSLAQWGASAAMQVVSRRQSEVRVPWLHNLAASQDESHDDQTDTEGEETDDEEEEEESEADENVLREVTALPNPRGLLGGVVHTVQNTLRNTISAVTWAPAAVLGTVGRILHLTPAQAVSSTKGRAMSLSDALKGVTDNVVDTVVHYVPLPRLSLMEPESEFRDIDNPSAEAERKGSGARPASPESTPRPGQPRGSLRSVRGLSAPSCPGLDDKTEASARPGFLAMPREKPARRVSDSFFRPSVMEPILGRAQYSQLRKKSSTVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 30 - DNA, 1305 bp, Homo sapiens
  CDS (1)..(1305)
atgtctgccgacggggcagaggctgatggcagcacccaggtgacagtggaagaaccggtacagcagcccagtgtggtggaccgtgtggccagcatgcctctgatcagctccacctgcgacatggtgtccgcagcctatgcctccaccaaggagagctacccgcacgtcaagactgtctgcgacgcagcagagaagggagtgaggaccctcacggcggctgctgtcagcggggctcagccgatcctctccaagctggagccccagattgcatcagccagcgaatacgcccacagggggctggacaagttggaggagaacctccccatcctgcagcagcccacggagaaggtcctggcggacaccaaggagcttgtgtcgtctaaggtgtcgggggcccaagagatggtgtctagcgccaaggacacggtggccacccaattgtcggaggcggtggacgcgacccgcggtgctgtgcagagcggcgtggacaagacaaagtccgtagtgaccggcggcgtccaatcagtcatgggctcccgcttgggccagatggtgctgagtggggtcgacacggtgctggggaagtcggaggagtgggcggacaaccacctgccccttacggatgccgaactggcccgcatcgccacatccctggatggcttcgacgtcgcgtccgtgcagcagcagcggcaggaacagagctacttcgtacgtctgggctccctgtcggagaggctgcggcagcacgcctatgagcactcgctgggcaagcttcgagccaccaagcagagggcacaggaggctctgctgcagctgtcgcaggccctaagcctgatggaaactgtcaagcaaggcgttgatcagaagctggtggaaggccaggagaagctgcaccagatgtggctcagctggaaccagaagcagctccagggccccgagaaggagccgcccaagccagagcaggtcgagtcccgggcgctcaccatgttccgggacattgcccagcaactgcaggccacctgtacctccctggggtccagcattcagggcctccccaccaatgtgaaggaccaggtgcagcaggcccgccgccaggtggaggacctccaggccacgttttccagcatccactccttccaggacctgtccagcagcattctggcccagagccgtgagcgtgtcgccagcgcccgcgaggccctggaccacatggtggaatatgtggcccagaacacacctgtcacgtggctcgtgggaccctttgcccctggaatcactgagaaagccccggaggagaagaaatag

SEQ ID NO: 31 - protein, 434 aa, Homo sapiens
MSADGAEADGSTQVTVEEPVQQPSVVDRVASMPLISSTCDMVSAAYASTKESYPHVKTVCDAAEKGVRTLTAAAVSGAQPILSKLEPQIASASEYAHRGLDKLEENLPILQQPTEKVLADTKELVSSKVSGAQEMVSSAKDTVATQLSEAVDATRGAVQSGVDKTKSVVTGGVQSVMGSRLGQMVLSGVDTVLGKSEEWADNHLPLTDAELARIATSLDGFDVASVQQQRQEQSYFVRLGSLSERLRQHAYEHSLGKLRATKQRAQEALLQLSQALSLMETVKQGVDQKLVEGQEKLHQMWLSWNQKQLQGPEKEPPKPEQVESRALTMFRDIAQQLQATCTSLGSSIQGLPTNVKDQVQQARRQVEDLQATFSSIHSFQDLSSSILAQSRERVASAREALDHMVEYVAQNTPVTWLVGPFAPGITEKAPEEKK

SEQ ID NO: 32 - DNA, 2058 bp, Artificial sequence
  fusion protein
atgcccgaggtagttttcggatcctctgccgacggggcagaggctgatggcagcacccaggtgacagtggaagaaccggtacagcagcccagtgtggtggaccgtgtggccagcatgcctctgatcagctccacctgcgacatggtgtccgcagcctatgcctccaccaaggagagctacccgcacgtcaagactgtctgcgacgcagcagagaagggagtgaggaccctcacggcggctgctgtcagcggggctcagccgatcctctccaagctggagccccagattgcatcagccagcgaatacgcccacagggggctggacaagttggaggagaacctccccatcctgcagcagcccacggagaaggtcctggcggacaccaaggagcttgtgtcgtctaaggtgtcgggggcccaagagatggtgtctagcgccaaggacacggtggccacccaattgtcggaggcggtggacgcgacccgcggtgctgtgcagagcggcgtggacaagacaaagtccgtagtgaccggcggcgtccaatcagtcatgggctcccgcttgggccagatggtgctgagtggggtcgacacggtgctggggaagtcggaggagtgggcggacaaccacctgccccttacggatgccgaactggcccgcatcgccacatccctggatggcttcgacgtcgcgtccgtgcagcagcagcggcaggaacagagctacttcgtacgtctgggctccctgtcggagaggctgcggcagcacgcctatgagcactcgctgggcaagcttcgagccaccaagcagagggcacaggaggctctgctgcagctgtcgcaggccctaagcctgatggaaactgtcaagcaaggcgttgatcagaagctggtggaaggccaggagaagctgcaccagatgtggctcagctggaaccagaagcagctccagggccccgagaaggagccgcccaagccagagcaggtcgagtcccgggcgctcaccatgttccgggacattgcccagcaactgcaggccacctgtacctccctggggtccagcattcagggcctccccaccaatgtgaaggaccaggtgcagcaggcccgccgccaggtggaggacctccaggccacgttttccagcatccactccttccaggacctgtccagcagcattctggcccagagccgtgagcgtgtcgccagcgcccgcgaggccctggaccacatggtggaatatgtggcccagaacacacctgtcacgtggctcgtgggaccctttgcccctggaatcactgagaaagccccggaggagaagaaaggatccagtacttctagagtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 33 - protein, 685 aa, Artificial sequence [sequence omitted]

SEQ ID NO: 34 - DNA, 1314 bp, Homo sapiens
  CDS (1)..(1314)
atggcatccgttgcagttgatccacaaccgagtgtggtgactcgggtggtcaacctgcccttggtgagctccacgtatgacctcatgtcctcagcctatctcagtacaaaggaccagtatccctacctgaagtctgtgtgtgagatggcagagaacggtgtgaagaccatcacctccgtggccatgaccagtgctctgcccatcatccagaagctagagccgcaaattgcagttgccaatacctatgcctgtaaggggctagacaggattgaggagagactgcctattctgaatcagccatcaactcagattgttgccaatgccaaaggcgctgtgactggggcaaaagatgctgtgacgactactgtgactggggccaaggattctgtggccagcacgatcacaggggtgatggacaagaccaaaggggcagtgactggcagtgtggagaagaccaagtctgtggtcagtggcagcattaacacagtcttggggagtcggatgatgcagctcgtgagcagtggcgtagaaaatgcactcaccaaatcagagctgttggtagaacagtacctccctctcactgaggaagaactagaaaaagaagcaaaaaaagttgaaggatttgatctggttcagaagccaagttattatgttagactgggatccctgtctaccaagcttcactcccgtgcctaccagcaggctctcagcagggttaaagaagctaagcaaaaaagccaacagaccatttctcagctccattctactgttcacctgattgaatttgccaggaagaatgtgtatagtgccaatcagaaaattcaggatgctcaggataagctctacctctcatgggtagagtggaaaaggagcattggatatgatgatactgatgagtcccactgtgctgagcacattgagtcacgtactcttgcaattgcccgcaacctgactcagcagctccagaccacgtgccacaccctcctgtccaacatccaaggtgtaccacagaacatccaagatcaagccaagcacatgggggtgatggcaggcgacatctactcagtgttccgcaatgctgcctcctttaaagaagtgtctgacagcctcctcacttctagcaaggggcagctgcagaaaatgaaggaatctttagatgacgtgatggattatcttgttaacaacacgcccctcaactggctggtaggtcccttttatcctcagctgactgagtctcagaatgctcaggaccaaggtgcagagatggacaagagcagccaggagacccagcgatctgagcataaaactcattaa

SEQ ID NO: 35 - protein, 437 aa, Homo sapiens
MASVAVDPQPSVVTRVVNLPLVSSTYDLMSSAYLSTKDQYPYLKSVCEMAENGVKTITSVAMTSALPIIQKLEPQIAVANTYACKGLDRIEERLPILNQPSTQIVANAKGAVTGAKDAVTTTVTGAKDSVASTITGVMDKTKGAVTGSVEKTKSVVSGSINTVLGSRMMQLVSSGVENALTKSELLVEQYLPLTEEELEKEAKKVEGFDLVQKPSYYVRLGSLSTKLHSRAYQQALSRVKEAKQKSQQTISQLHSTVHLIEFARKNVYSANQKIQDAQDKLYLSWVEWKRSIGYDDTDESHCAEHIESRTLAIARNLTQQLQTTCHTLLSNIQGVPQNIQDQAKHMGVMAGDIYSVFRNAASFKEVSDSLLTSSKGQLQKMKESLDDVMDYLVNNTPLNWLVGPFYPQLTESQNAQDQGAEMDKSSQETQRSEHKTH

SEQ ID NO: 36 - DNA, 2067 bp, Artificial sequence
  fusion protein
atgcccgaggtagttttcggatccagtactgcatccgttgcagttgatccacaaccgagtgtggtgactcgggtggtcaacctgcccttggtgagctccacgtatgacctcatgtcctcagcctatctcagtacaaaggaccagtatccctacctgaagtctgtgtgtgagatggcagagaacggtgtgaagaccatcacctccgtggccatgaccagtgctctgcccatcatccagaagctagagccgcaaattgcagttgccaatacctatgcctgtaaggggctagacaggattgaggagagactgcctattctgaatcagccatcaactcagattgttgccaatgccaaaggcgctgtgactggggcaaaagatgctgtgacgactactgtgactggggccaaggattctgtggccagcacgatcacaggggtgatggacaagaccaaaggggcagtgactggcagtgtggagaagaccaagtctgtggtcagtggcagcattaacacagtcttggggagtcggatgatgcagctcgtgagcagtggcgtagaaaatgcactcaccaaatcagagctgttggtagaacagtacctccctctcactgaggaagaactagaaaaagaagcaaaaaaagttgaaggatttgatctggttcagaagccaagttattatgttagactgggatccctgtctaccaagcttcactcccgtgcctaccagcaggctctcagcagggttaaagaagctaagcaaaaaagccaacagaccatttctcagctccattctactgttcacctgattgaatttgccaggaagaatgtgtatagtgccaatcagaaaattcaggatgctcaggataagctctacctctcatgggtagagtggaaaaggagcattggatatgatgatactgatgagtcccactgtgctgagcacattgagtcacgtactcttgcaattgcccgcaacctgactcagcagctccagaccacgtgccacaccctcctgtccaacatccaaggtgtaccacagaacatccaagatcaagccaagcacatgggggtgatggcaggcgacatctactcagtgttccgcaatgctgcctcctttaaagaagtgtctgacagcctcctcacttctagcaaggggcagctgcagaaaatgaaggaatctttagatgacgtgatggattatcttgttaacaacacgcccctcaactggctggtaggtcccttttatcctcagctgactgagtctcagaatgctcaggaccaaggtgcagagatggacaagagcagccaggagacccagcgatctgagcataaaactcatagtacttctagagtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 37 - protein, 688 aa, Artificial sequence [sequence omitted]

SEQ ID NO: 38 - DNA, 564 bp, Maize
  CDS (1)..(564)
atggcggaccgtgaccgcagcggcatctacggcggcgcccacgccacctacgggcagcagcagcagcagggaggaggcgggcgcccgatgggtgagcaggtgaaaaagggcatgctccacgacaaggggccgacggcgtcgcaggcgctgacggtggcgacgctgttcccgctgggcgggctgctgctggtgctgtcggggctggcgctgacggcctccgtggtggggctggccgtggccacgccggtgttcctgatcttcagccccgtgctggtccccgccgcgctgctcatcgggacggccgtcatggggttcctcacgtcgggcgcgctggggctcgggggcctgtcctcgctcacgtgcctcgccaacacggcgcggcaggcgttccagcgcaccccggactacgtggaggaggcgcgccgcaggatggcggaggccgcggcgcaagcgggccacaagaccgcgcaggcaggccaggccatccagggcagggcgcaggaggccggcaccgggggaggtgcaggtgccggcgctggcggcggcggcagggcttcctcgtaa

SEQ ID NO: 39 - protein, 187 aa, Maize
MADRDRSGIYGGAHATYGQQQQQGGGGRPMGEQVKKGMLHDKGPTASQALTVATLFPLGGLLLVLSGLALTASVVGLAVATPVFLIFSPVLVPAALLIGTAVMGFLTSGALGLGGLSSLTCLANTARQAFQRTPDYVEEARRRMAEAAAQAGHKTAQAGQAIQGRAQEAGTGGGAGAGAGGGGRASS

SEQ ID NO: 40 - DNA, 1317 bp, Artificial sequence
  fusion protein
atgcccgaggtagttttcggatccgcggaccgtgaccgcagcggcatctacggcggcgcccacgccacctacgggcagcagcagcagcagggaggaggcgggcgcccgatgggtgagcaggtgaaaaagggcatgctccacgacaaggggccgacggcgtcgcaggcgctgacggtggcgacgctgttcccgctgggcgggctgctgctggtgctgtcggggctggcgctgacggcctccgtggtggggctggccgtggccacgccggtgttcctgatcttcagccccgtgctggtccccgccgcgctgctcatcgggacggccgtcatggggttcctcacgtcgggcgcgctggggctcgggggcctgtcctcgctcacgtgcctcgccaacacggcgcggcaggcgttccagcgcaccccggactacgtggaggaggcgcgccgcaggatggcggaggccgcggcgcaagcgggccacaagaccgcgcaggcaggccaggccatccagggcagggcgcaggaggccggcaccgggggaggtgcaggtgccggcgctggcggcggcggcagggcttcctcgggatccagtacttctagagtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 41 - protein, 438 aa, Artificial sequence
  Synthetic Construct
MPEVVFGSADRDRSGIYGGAHATYGQQQQQGGGGRPMGEQVKKGMLHDKGPTASQALTVATLFPLGGLLLVLSGLALTASVVGLAVATPVFLIFSPVLVPAALLIGTAVMGFLTSGALGLGGLSSLTCLANTARQAFQRTPDYVEEARRRMAEAAAQAGHKTAQAGQAIQGRAQEAGTGGGAGAGAGGGGRASSGSSTSRVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 42 - DNA, 900 bp, Escherichia coli
  CDS (1)..(900)
atggactttccgcagcaactcgaagcctgcgttaagcaggccaaccaggcgctgagccgttttatcgccccactgccctttcagaacactcccgtggtcgaaaccatgcagtatggcgcattattaggtggtaagcgcctgcgacctttcctggtttatgccaccggtcatatgttcggcgttagcacaaacacgctggacgcacccgctgccgccgttgagtgtatccacgcttactcattaattcatgatgatttaccggcaatggatgatgacgatctgcgtcgcggtttgccaacctgccatgtgaagtttggcgaagcaaacgcgattctcgctggcgacgctttacaaacgctggcgttctcgattttaagcgatgccgatatgccggaagtgtcggaccgcgacagaatttcgatgatttctgaactggcgagcgccagtggtattgccggaatgtgcggtggtcaggcattagatttagacgcggaaggcaaacacgtacctctggacgcgcttgagcgtattcatcgtcataaaaccggcgcattgattcgcgccgccgttcgccttggtgcattaagcgccggagataaaggacgtcgtgctctgccggtactcgacaagtatgcagagagcatcggccttgccttccaggttcaggatgacatcctggatgtggtgggagatactgcaacgttgggaaaacgccagggtgccgaccagcaacttggtaaaagtacctaccctgcacttctgggtcttgagcaagcccggaagaaagcccgggatctgatcgacgatgcccgtcagtcgctgaaacaactggctgaacagtcactcgatacctcggcactggaagcgctagcggactacatcatccagcgtaataaataa

SEQ ID NO: 43 - protein, 299 aa, Escherichia coli
MDFPQQLEACVKQANQALSRFIAPLPFQNTPVVETMQYGALLGGKRLRPFLVYATGHMFGVSTNTLDAPAAAVECIHAYSLIHDDLPAMDDDDLRRGLPTCHVKFGEANAILAGDALQTLAFSILSDADMPEVSDRDRISMISELASASGIAGMCGGQALDLDAEGKHVPLDALERIHRHKTGALIRAAVRLGALSAGDKGRRALPVLDKYAESIGLAFQVQDDILDVVGDTATLGKRQGADQQLGKSTYPALLGLEQARKKARDLIDDARQSLKQLAEQSLDTSALEALADYIIQRNK

SEQ ID NO: 44 - DNA, 930 bp, Erwinia uredovora
  CDS (1)..(930)
atgaataatccgtcgttactcaatcatgcggtcgaaacgatggcagttggctcgaaaagttttgcgacagcctcaaagttatttgatgcaaaaacccggcgcagcgtactgatgctctacgcctggtgccgccattgtgacgatgttattgacgatcagacgctgggctttcaggcccggcagcctgccttacaaacgcccgaacaacgtctgatgcaacttgagatgaaaacgcgccaggcctatgcaggatcgcagatgcacgaaccggcgtttgcggcttttcaggaagtggctatggctcatgatatcgccccggcttacgcgtttgatcatctggaaggcttcgccatggatgtacgcgaagcgcaatacagccaactggatgatacgctgcgctattgctatcacgttgcaggcgttgtcggcttgatgatggcgcaaatcatgggcgtgcgggataacgccacgctggaccgcgcctgtgaccttgggctggcatttcagttgaccaatattgctcgcgatattgtggacgatgcgcatgcgggccgctgttatctgccggcaagctggctggagcatgaaggtctgaacaaagagaattatgcggcacctgaaaaccgtcaggcgctgagccgtatcgcccgtcgtttggtgcaggaagcagaaccttactatttgtctgccacagccggcctggcagggttgcccctgcgttccgcctgggcaatcgctacggcgaagcaggtttaccggaaaataggtgtcaaagttgaacaggccggtcagcaagcctgggatcagcggcagtcaacgaccacgcccgaaaaattaacgctgctgctggccgcctctggtcaggcccttacttcccggatgcgggctcatcctccccgccctgcgcatctctggcagcgcccgctctag

SEQ ID NO: 45 - protein, 309 aa, Erwinia uredovora
MNNPSLLNHAVETMAVGSKSFATASKLFDAKTRRSVLMLYAWCRHCDDVIDDQTLGFQARQPALQTPEQRLMQLEMKTRQAYAGSQMHEPAFAAFQEVAMAHDIAPAYAFDHLEGFAMDVREAQYSQLDDTLRYCYHVAGVVGLMMAQIMGVRDNATLDRACDLGLAFQLTNIARDIVDDAHAGRCYLPASWLEHEGLNKENYAAPENRQALSRIARRLVQEAEPYYLSATAGLAGLPLRSAWAIATAKQVYRKIGVKVEQAGQQAWDQRQSTTTPEKLTLLLAASGQALTSRMRAHPPRPAHLWQRPL

SEQ ID NO: 46 - DNA, 909 bp, Erwinia uredovora
  CDS (1)..(909)
atgacggtctgcgcaaaaaaacacgttcatctcactcgcgatgctgcggagcagttactggctgatattgatcgacgccttgatcagttattgcccgtggagggagaacgggatgttgtgggtgccgcgatgcgtgaaggtgcgctggcaccgggaaaacgtattcgccccatgttgctgttgctgaccgcccgcgatctgggttgcgctgtcagccatgacggattactggatttggcctgtgcggtggaaatggtccacgcggcttcgctgatccttgacgatatgccctgcatggacgatgcgaagctgcggcgcggacgccctaccattcattctcattacggagagcatgtggcaatactggcggcggttgccttgctgagtaaagcctttggcgtaattgccgatgcagatggcctcacgccgctggcaaaaaatcgggcggtttctgaactgtcaaacgccatcggcatgcaaggattggttcagggtcagttcaaggatctgtctgaaggggataagccgcgcagcgctgaagctattttgatgacgaatcactttaaaaccagcacgctgttttgtgcctccatgcagatggcctcgattgttgcgaatgcctccagcgaagcgcgtgattgcctgcatcgtttttcacttgatcttggtcaggcatttcaactgctggacgatttgaccgatggcatgaccgacaccggtaaggatagcaatcaggacgccggtaaatcgacgctggtcaatctgttaggcccgagggcggttgaagaacgtctgagacaacatcttcagcttgccagtgagcatctctctgcggcctgccaacacgggcacgccactcaacattttattcaggcctggtttgacaaaaaactcgctgccgtcagttaa

SEQ ID NO: 47 - protein, 302 aa, Erwinia uredovora
MTVCAKKHVHLTRDAAEQLLADIDRRLDQLLPVEGERDVVGAAMREGALAPGKRIRPMLLLLTARDLGCAVSHDGLLDLACAVEMVHAASLILDDMPCMDDAKLRRGRPTIHSHYGEHVAILAAVALLSKAFGVIADADGLTPLAKNRAVSELSNAIGMQGLVQGQFKDLSEGDKPRSAEAILMTNHFKTSTLFCASMQMASIVANASSEARDCLHRFSLDLGQAFQLLDDLTDGMTDTGKDSNQDAGKSTLVNLLGPRAVEERLRQHLQLASEHLSAACQHGHATQHFIQAWFDKKLAAVS

SEQ ID NO: 48 - DNA, 1479 bp, Erwinia uredovora
  CDS (1)..(1479)
atgaaaccaactacggtaattggtgcaggcttcggtggcctggcactggcaattcgtctacaagctgcggggatccccgtcttactgcttgaacaacgtgataaacccggcggtcgggcttatgtctacgaggatcaggggtttacctttgatgcaggcccgacggttatcaccgatcccagtgccattgaagaactgtttgcactggcaggaaaacagttaaaagagtatgtcgaactgctgccggttacgccgttttaccgcctgtgttgggagtcagggaaggtctttaattacgataacgatcaaacccggctcgaagcgcagattcagcagtttaatccccgcgatgtcgaaggttatcgtcagtttctggactattcacgcgcggtgtttaaagaaggctatctaaagctcggtactgtcccttttttatcgttcagagacatgcttcgcgccgcacctcaactggcgaaactgcaggcatggagaagcgtttacagtaaggttgccagttacatcgaagatgaacatctgcgccaggcgttttctttccactcgctgttggtgggcggcaatcccttcgccacctcatccatttatacgttgatacacgcgctggagcgtgagtggggcgtctggtttccgcgtggcggcaccggcgcattagttcaggggatgataaagctgtttcaggatctgggtggcgaagtcgtgttaaacgccagagtcagccatatggaaacgacaggaaacaagattgaagccgtgcatttagaggacggtcgcaggttcctgacgcaagccgtcgcgtcaaatgcagatgtggttcatacctatcgcgacctgttaagccagcaccctgccgcggttaagcagtccaacaaactgcagactaagcgcatgagtaactctctgtttgtgctctattttggtttgaatcaccatcatgatcagctcgcgcatcacacggtttgtttcggcccgcgttaccgcgagctgattgacgaaatttttaatcatgatggcctcgcagaggacttctcactttatctgcacgcgccctgtgtcacggattcgtcactggcgcctgaaggttgcggcagttactatgtgttggcgccggtgccgcatttaggcaccgcgaacctcgactggacggttgaggggccaaaactacgcgaccgtatttttgcgtaccttgagcagcattacatgcctggcttacggagtcagctggtcacgcaccggatgtttacgccgtttgattttcgcgaccagcttaatgcctatcatggctcagccttttctgtggagcccgttcttacccagagcgcctggtttcggccgcataaccgcgataaaaccattactaatctctacctggtcggcgcaggcacgcatcccggcgcaggcattcctggcgtcatcggctcggcaaaagcgacagcaggtttgatgctggaggatctgatatga

SEQ ID NO: 49 - protein, 492 aa, Erwinia uredovora
MKPTTVIGAGFGGLALAIRLQAAGIPVLLLEQRDKPGGRAYVYEDQGFTFDAGPTVITDPSAIEELFALAGKQLKEYVELLPVTPFYRLCWESGKVFNYDNDQTRLEAQIQQFNPRDVEGYRQFLDYSRAVFKEGYLKLGTVPFLSFRDMLRAAPQLAKLQAWRSVYSKVASYIEDEHLRQAFSFHSLLVGGNPFATSSIYTLIHALEREWGVWFPRGGTGALVQGMIKLFQDLGGEVVLNARVSHMETTGNKIEAVHLEDGRRFLTQAVASNADVVHTYRDLLSQHPAAVKQSNKLQTKRMSNSLFVLYFGLNHHHDQLAHHTVCFGPRYRELIDEIFNHDGLAEDFSLYLHAPCVTDSSLAPEGCGSYYVLAPVPHLGTANLDWTVEGPKLRDRIFAYLEQHYMPGLRSQLVTHRMFTPFDFRDQLNAYHGSAFSVEPVLTQSAWFRPHNRDKTITNLYLVGAGTHPGAGIPGVIGSAKATAGLMLEDLI

(19) United States
(12) Patent Application Publication
(10) Pub. No.: US 2009/0203093 A1
(43) Pub. Date: Aug. 13, 2009

(54) Protein Targeting To Lipid Bodies

(75) Inventors: Alexander Steinbüchel, Altenberge (DE); Marc Wältermann, Offenbach am Main (DE); Jan Hänisch, Braunschweig (DE)

Correspondence Address: CONNOLLY BOVE LODGE & HUTZ, LLP, P O BOX 2207, WILMINGTON, DE, 19899, US

(73) Assignee: BASF SE, Ludwigshafen (DE)

(21) Appl. No.: 12/307,690

(22) PCT Filed: Jul. 10, 2007
(86) PCT No.: PCT/EP2007/057047

§ 371 Date: Jan. 6, 2009

(30) Foreign Application Priority Data
Jul. 11, 2006 (EP) 06117001.5

(51) Int. Cl. C12P 7/64; C12N 15/87; C07K 14/00; C12N 15/11; C12N 15/00; C12N 1/21

(52) U.S. Cl. 435/134; 435/471; 530/350; 536/23.4; 435/320.1; 435/252.3; 435/252.31; 435/252.32; 435/252.33; 435/252.35

(57) ABSTRACT

The present invention relates to a method of targeting a protein of interest to an intracellular hydrophobic inclusion body of a bacterial cell by means of a fusion protein comprising a hydrophobic targeting peptide operatively linked with said protein of interest; methods of microbial production of a lipophilic compound of interest by means of a recombinant bacterial host comprising intracellular inclusion bodies having at least one enzyme which is involved in the biosynthesis of said lipophilic compound targeted to said inclusion bodies; as well as corresponding fusion proteins, coding sequences, expression vectors and recombinant hosts.

The present invention relates to a method of targeting a protein of interest to an intracellular hydrophobic inclusion body of a bacterial cell by means of a fusion protein comprising a hydrophobic targeting peptide operatively linked with said protein of interest; methods of microbial production of a lipophilic compound of interest by means of a recombinant bacterial host comprising intracellular inclusion bodies having at least one enzyme which is involved in the biosynthesis of said lipophilic compound targeted to said inclusion bodies; as well as corresponding fusion proteins, coding sequences, expression vectors and recombinant hosts.

### BACKGROUND OF THE INVENTION

Most organisms are capable to accumulate hydrophobic compounds, such as triacylglycerols (TAGs), wax esters (WEs), sterols esters or poly(hydroxyalkanoates) (PHAs). These lipids and polymers are deposited as intracellular inclusions and serve mainly as energy and carbon reserves or precursors for membrane lipid and steroid biosynthesis.

The primary energy storage compounds in eukaryotes are TAGs, whereas most prokaryotes synthesize PHAs [18, 24]. In bacteria, reserve TAGs and WEs are mainly restricted to nocardioform actinomycetes, streptomycetes and some Gram-negative strains [3, 31]. As the most prominent example, Ralstonia eutropha H16 is capable to accumulate poly(3-hydroxybutyrate) (PHB) up to 90% of its cell dry weight (Steinbüchel, [24]).

Bacterial neutral lipid inclusions are structurally related to those in eukaryotes. Both consist of a lipid core surrounded by a monolayer of phospholipids, which shield the inclusions from the cytoplasm, thereby preventing coalescence or denaturation of cytoplasmic proteins due to hydrophobic interactions.

The biogenesis and protein equipment of TAG and WE inclusions in bacteria differ significantly from eukaryotic lipid inclusions. In eukaryotes, lipid inclusions are assumed to emanate by accumulation of lipids between both phospholipid leaflets at the endoplasmic reticulum (ER) and subsequent lipid body budding. The budding particle, which has a phospholipid monolayer membrane derived from the outer ER leaflet, is finally released into the cytoplasm [5, 18]. In contrast, in bacteria TAGs and WEs are synthesized by wax ester synthase/acyl-CoA:diacylglycerol acyltransferase (WS/DGAT) as small enzyme bound droplets at the cytoplasmic face of the plasma membrane. These droplets aggregate to larger structures, which are assumed to be coated by phospholipids, before they are released into the cytoplasm [11, 30].

Whereas in animals and most plants the lipid body monolayer is associated with embedded proteins, no such proteins are known to surround bacterial lipid inclusions [12, 30]. The perilipins are the best characterized mammalian lipid body proteins and are involved in structure and formation of the organelles and control of lipid balance, by regulating lipolysis by hormone-sensitive lipase [17]. Three perilipin isoforms, A, B, and C, are encoded by alternatively spliced forms of mRNA transcribed from a single gene [9, 16]. All perilipins share a common N-terminus, which is also very similar to that of ADRP and TIP47, which together constitute the PAT protein family [15]. Perilipin A is the largest isoform and the most abundant protein associated with adipocyte lipid bodies, whereas ADRP and TIP47 have a broad tissue distribution. Perilipins and ADRP are specifically associated with the lipid body surface, whereas TIP47 is also abundant in the cytoplasm [4, 17]. Reports on whether PAT family proteins are synthesized on free ribosomes or are cotranslationally inserted into nascent lipid bodies along the ER, similar to oleosins in plants, are contradictory [5, 8, 15, 20].

Oleosins [1,13] are the main proteins which are associated with lipid bodies in the seeds of dessication tolerant plants. They are assumed to play a key role in the maintenance of stability of the lipid bodies, since they prevent them to coalesce during seed dehydration and germination [18]. Oleosins are assumed to be synthesized by polyribosomes on the ER and incorporated cotranslationally into lipid bodies during the budding process. This ER-mediated targeting appears to be universal in eukaryotes, since oleosins from maize have also been successfully targeted to seed lipid bodies in Brassica napus, and also in recombinant yeast (Saccharomyces cerevisiae) [14, 26].

There were also large differences revealed regarding the protein composition and formation between prokaryotic PHA inclusions on one side and prokaryotic WE or TAG inclusions on the other side. Whereas no specific proteins are known to be abundantly associated with bacterial TAG and WE inclusions, PHA inclusions are coated by phasins, which represent a unique class of proteins (Pötter & Steinbüchel [18c]; Wältermann & Steinbüchel [31]; Steinbüchel et al. [24b]). PhaP1, which represents the major phasin on the surface of PHA inclusions in R. eutropha H16, plays an important role in the formation and structure of these inclusions, because its presence or absence affects the number and size of the inclusions and the amount of PHB in the cells (Wieczorek et al. [31a], Pötter et al., [18d], Pötter et al. [18e], York et al. [32]). According to the most accepted model, PHA inclusions are formed from soluble PHA synthases polymerizing 3-hydroxybutyrate (3HB) of 3HB-CoA to PHB with concomitant release of CoA. Since PHA synthases remain covalently linked to the growing PHB chain, an amphiphilic complex composed of the hydrophilic synthase and the elongating polymer chain is formed (Gerngross et al., [8a]). These complexes are thought to aggregate to micelle-like structures, which enlarge to PHA granules due to proceeding extension of the PHA chains. During granule growth, phasins and phospholipids are thought to immigrate to the exposed hydrophobic surface of the polymer core, thereby generating an interphase between the hydrophobic core and the cytoplasm (Stubbe & Tian, [24c]). However, no three-dimensional structures of phasin proteins have been reported, yet, and little is known about the factors and motifs mediating and influencing their targeting to PHA granules (Pieper-Fürst et al. [18b]).

In contrast to this and as already described above, TAGs and WEs are formed at the cytoplasmic site of the plasma membrane by wax ester synthase/acyl-CoA:diacylglycerol acyltransferase (WS/DGAT). The latter is the key enzyme for biosynthesis of these lipids in bacteria and is bound to lipid droplets. These small droplets coalesce to larger structures which are then released into the cytoplasm and appear finally as large lipid inclusions.

There is need for systems allowing the targeting of functional polypeptides, as for example functional enzymes, to the lipid bodies, as for example TAGs, as formed by bacterial cells, which remain associated with said lipid bodies for a sufficient time in order to make use of their functionality within said cells.

### SUMMARY OF THE INVENTION

The above-mentioned problem was surprisingly solved by transforming lipid body producing bacterial cells with the coding sequence for a fusion protein comprising a targeting peptide operably linked with a functional polypeptide, as for example a functional enzyme.

### DESCRIPTION OF FIGURES

FIG. 1: (A) Effect of acetamide induction on synthesis of PhaP1, eGFP and the C-terminal PhaP1-eGFP fusion in M. smegmatis harbouring the constructed expression plasmids by employing SDS-PAGE (left) and immunological detection of the respective recombinant proteins by employing Western blot analysis (right). Antibodies used for the detection of the respective proteins were indicated in the figure. Std, molecular weight standard; lane 1, M. smegmatis pJAM2::phaP1 in the absence of acetamide; M. smegmatis lane 2, pJAM2::phaP1 induced with 0.5% (w/v) acetamide; lane 3, M. smegmatis pJAM2::egfp in the absence of acetamide; lane 4, M. smegmatis pJAM2::egfp induced with 0.5% (w/v) acetamide; lane 5; M. smegmatis pJAM2::phaP1-egfp in the absence of acetamide; lane 6, pJAM2::phaP1-egfp induced with 0.5% (w/v) acetamide. (B) Time course analysis of recombinant PhaP1 synthesis and stability in M. smegmatis harbouring pJAM2::phaP1. Electropherograms (left) of cell crude extracts and immunological detection of PhaP1 by employing anti-PhaP1 IgGs on Western blot corresponding to the SDS-PAGE (right) after 24 (lane 1), 48 (lane 2), 72 (lane 3) and 96 h (lane 4) of growth in ammonium reduced MSM supplemented with 0.5% (w/v) acetamide. Proteins in the SDS-PAGE gels presented in (A) and (B) were visualized by Coomassie Brilliant Blue R250 (C) Effect of different concentrations of acetamide on intracellular TAG accumulation in M. smegmatis after 72 h growth in ammonium reduced MSM as revealed by TLC. Std, triolein standard; lane 1, 0.5% (w/v); lane 2, 0.3% (w/v); lane 3, 0.1% (w/v); lane 4, 0.05% (w/v); lane 5, 0.01% (w/v); lane 6, 0.005% (w/v), lane 7, 0.001% (w/v).

FIG. 2: Immunological detection of PhaP1, eGFP and the PhaP1-eGFP fusion in cell crude extracts and subcellular fractions obtained from R. opacus wild type cells and respective recombinant strains harbouring plasmids pJAM2::phaP1, pJAM2::egfp or pJAM2::phaP1-egfp. Left image shows SDS-PAGE electropherograms of the crude extracts and cellular fractions, whereas the images in the center and on the right show the immunological assays by employing anti-PhaP1 IgGs and anti-eGFP IgGs on Western blots corresponding to the SDS-PAGE, respectively. Proteins in the gel were stained with Coomassie Brilliant Blue R250. Std, Molecular weight standard; lane 1; crude extract of wild type cells; lane 2, soluble fraction of wild type cells; lane 3, TAG inclusions isolated from wild type cells; lane 4, crude extract of cells harbouring pJAM2::phaP1; lane 5, soluble fraction obtained from cells harbouring pJAM2::phaP1; lane 6; TAG inclusions isolated from cells harbouring pJAM2::phaP1; lane 7, crude extract of cells harbouring pJAM2::egfp; lane 8, soluble fraction of cells harbouring pJAM2::egfp; lane 9, TAG inclusions of cells harbouring pJAM2::egfp; lane 10, cell crude extract of cells harbouring pJAM2::phaP1-egfp; lane 11, soluble fraction of cells harbouring pJAM2::phaP1-egfp; lane 12, TAG inclusions isolated from pJAM2::phaP1-egfp harbouring cells. Cells were grown 72 h in ammonium reduced MSM supplemented with 0.5% (w/v) acetamide.

FIG. 3: Fluorescence microscopic localization of Nile Red and the PhaP1-eGFP fusion in recombinant cells of R. opacus grown in (A) Std1 medium and for 24 (B), 48 (C) or 72 h (D) in ammonium reduced MSM. Images at the top of each panel show phase contrast (PH), differential interference contrast (DIC) and three channel fluorescence microscopic overlay images merged from PH, Nile Red- (NR) and eGFP-fluorescent images. Images at the bottom of each panel show single channel eGFP and NR images and a two channel fluorescence microscopic overlay image merged from NR and eGFP fluorescence. In addition, panel A shows a deconvoluted image of R. opacus grown in Std1 revealing slight PhaP1-eGFP fluorescence at the cytoplasm membrane (arrow), whereas the additional deconvoluted image in panel D demonstrates PhaP1-eGFP fluorescence at the surface of intracellular TAG inclusions in a cell grown for 72 h in ammonium reduced MSM. (E) A PH and deconvoluted two-channel eGFP/NR fluorescent image of a TAG inclusion isolated from a phaP1-egfp expressing R. opacus cell grown for 72 h under storage conditions showing a distribution of the fusion protein at the surface and a labeling of the lipids in the core of the inclusion by NR. (F) PH and fluorescence images of cells of R. opacus transformed with pJAM2::egfp grown for 48 h under storage conditions showing a diffuse cytoplasmic fluorescence of unfused eGFP (upper panel), whereas intracellular TAG inclusions were clearly labeled by NR in a two channel eGFP/NR fluorescent image (lower panel). All images were obtained from cells cultivated in the presence of 0.5% (w/v) acetamide. Bars represent 1 μm if not otherwise stated.

FIG. 4: Fluorescence microscopic localization of PhaP1-eGFP fusion protein in recombinant cells of M. smegmatis mc²155. Images at the left show phase contrast images, whereas images at the right show the corresponding fluorescence images. Cells of the control strain harbouring pJAM2::egfp show diffuse fluorescence of the unfused eGFP throughout the cytoplasm (A). A cell of M. smegmatis mc²155 transformed with pJAM2::phaP1 grown in Std1 medium exhibiting a single, fluorescent TAG inclusion at one of its cell pole (B). Cells harbouring pJAM2::phaP1-egfp grown in ammonium reduced MSM for 24 h (C) and 48 h (D) showing increased numbers of TAG inclusions tagged with PhaP1-eGFP (arrow). All images were obtained from cells cultivated in the absence of acetamide.

FIG. 5: PhaP1 is associated with intracellular TAG inclusions and the plasma membrane in recombinant R. opacus PD630. Immunocytochemistry was done on a cryosection applying rabbit anti-PhaP1 IgGs followed by 18 nm gold conjugated goat anti-rabbit pig IgGs (black dots). Cells were transformed with pJAM2::phaP1 and grown for 72 h under storage conditions before preparation of sections was done as described in the Methods section. Abbreviations: CW, cell wall; CY, cytoplasm; TAG, TAG inclusion; Scale bar=200 nm.

FIG. 6: β-Galactosidase activities of isolated TAG inclusions isolated from cells of recombinant R. opacus PD630. (A) β-Galactosidase activity of TAG inclusions isolated from cells of R. opacus PD630 harbouring pJAM2::phaP1-lacZ. (B) β-Galactosidase activity of assay (A) after removing TAG inclusions by filtration as described in the Methods section. (C) β-Galactosidase activity of TAG inclusions isolated from cells of R. opacus PD630 harbouring pJAM2::phaP1 as a control. (D) β-Galactosidase activity of assay (C) after removal of TAG inclusions.

FIG. 7: Immunological detection of maize oleosins and murine perilipin A expression in crude protein extracts of recombinant cells of M. smegmatis mc²155. (A) SDS-PAGE: Std, Molecular weight standard; lane 1 and 3, M. smegmatis pJAM2; lane 2, M. smegmatis pJAM2::oleo_{mays}; lane 4, M. smegmatis pJAM2::perAₘᵤᵣ.

(B and C) Immunoblot detection of maize oleosin (B) and murine perlipin A (C) corresponding to the SDS-PAGE (A).

FIG. 8: Distribution of eGFP fusion of perilipin A in recombinant R. opacus PD630. Left panel shows phase contrast images whereas the right side shows the corresponding fluorescence images.

(A) A pJAM2::egfp transformed cell of R. opacus PD630 grown for 24 h under storage conditions shows a diffuse cytoplasmic fluorescence of unfused eGFP. Arrow indicates an area excluded from fluorescence due to an intracellular, unmarked TAG inclusion. (B) Cells transformed with pJAM2::perAₘᵤᵣ-egfp grown for 0, 24 or 48 h, respectively, under storage conditions expressing eGFP fused murine perilipin A. Fluorescence of the eGFP fusion is associated with intracellular TAG inclusions (arrow). (C) TAG inclusion isolated from a perilipin A-eGFP expressing R. opacus PD630 cell grown for 48 h under storage conditions. After isolation of the TAG inclusion, counterstaining of core lipids was performed with Nile Red.

FIG. 9: Distribution of eGFP fusion of TIP47 in recombinant R. opacus PD630. Phase contrast images are depicted on the left panel and corresponding fluorescence images on the right panel. (A) Time lap experiment demonstrating the formation of intracellular TAG inclusions and association of TIP47-eGFP protein with these inclusions in recombinant R. opacus PD630. (B) Isolated TAG inclusion contrasted with Nile Red carrying associated TIP47-eGFP fusions. TAG inclusions were isolated from cultured cells grown for 48 h under lipid storage conditions.

FIG. 10: Distribution of eGFP fusion of ADRP in recombinant R. opacus PD630. Phase contrast images (left panel) and corresponding fluorescence images (right panel) are shown. Cells were transformed with pJAM2::adrpₕᵤₘ-egfp and grown for 0, 24 and 48 h under storage condition.

FIG. 11: Immunogold labeling of TIP47 in cytoplasmic TAG inclusions of cryosectioned and freeze-fractured recombinant R. opacus PD630 cells. (A) Immunogold labeling of TIP47 on a cryosection applying guinea pig anti-human IgGs followed by 18 nm gold conjugated donkey anti-guinea pig IgGs. Cells were transformed with pJAM2::tip47 and grown for 24 h under storage conditions. Immunogold (12 nm gold) labeling of the fusion protein of its TIP47 portion over the cores of intracellular TAG inclusions in concavely (B) and convexly (C) fractured cells. (D) Immunogold (12 nm gold) labeling of TIP47-eGFP by means of their eGFP-tag in the cores of cross-fractured TAG inclusions. Abbreviations: Cw, cell wall; Cy, cytoplasma; TAG, TAG inclusions. Bars=200 nm.

### DETAILED DESCRIPTION

### 1. Preferred Embodiments

In a first aspect, the present invention relates to a method of targeting a protein of interest to an intracellular hydrophobic inclusion body of a recombinant bacterial cell, which method comprises heterologously expressing in said bacterial cell a nucleotide sequence encoding a fusion protein comprising a hydrophobic targeting peptide operatively linked with said protein of interest.

In general, said inclusion bodies are of the TAG-, WE- or PHA-type. Preferably they are TAG-inclusion bodies.

The targeting peptide as used in the present method is selected from pro- or eukaryotic peptides and is in particular selected from peptides of bacterial, animal or plant origin. Preferred are targeting molecules of bacterial and animal origin. In particular, the targeting molecule is either derived from a protein associated in its native state with prokaryotic in particular bacterial PHA inclusion bodies; or is derived from a protein associated in its native state with eukaryotic, in particular animal or plant TAG or WE inclusion bodies.

As specific classes of targeting molecules there may be mentioned polyhydroxyalkanoate body binding phasins as for example PhaP1; Members of the PAT family of targeting proteins, in particular: perilipins, as for example perilipin A, B or C; Adipose Differentiation Related Proteins (ADRPs) also known as adipophilins; and Tail Interacting Proteins (TIPs) as for example TIP47. Non-limiting examples of targeting molecules are selected from:

### a) PhaP1 (SEQ ID NO:19)

### b) Perilipin A (SEQ ID NO:27)

### c) ADRP (SEQ ID NO:35)

### d) TIP47 (SEQ ID NO:31)

or functional equivalents thereof.

In a preferred embodiment of the targeting method said protein of interest is an enzyme, as for example an enzyme involved in the biosynthesis of hydrophobic or lipophilic compounds of interest. For example, said enzyme may be involved in the biosynthesis of
a) lipophilic vitamins, derivatives and precursors thereof,
b) saturated or unsaturated fatty acids and fatty alcohols, in particular long-chain fatty acids or corresponding fatty alcohols having 10 to 30 or 18 to 25 carbon atoms, as for example polyunsaturated fatty acids (PUFAs) or
c) flavouring substances.

As non-limiting examples of group a) compounds the may be mentioned carotenoids as for example β-carotene, lutein, lycopene, cantaxanthine, zeaxanthine, astaxantine; vitamins as for example vitamin E and Q10.

As non-limiting examples of group b) compounds the may be mentioned PUFAs havon 18 to 22 carbon atoms and 3 to 6 C═C-bonds, as for example the omega-3 fatty acids: 18:3ω3, 18:4ω3, 20:3ω3, 20:4ω3, 20:5ω3 (i.e. eicosapentaenoic acid, EPA), 22:5ω3, 22:6ω3 (i.e. docosahexaenoic acid, DHA); or omega-6 fatty acids: 18:2ω6, 18:3ω6, 20:2ω6, 20:3ω6 (i.e. bishomo-gamma-linolenic acid, DGLA), 20:4ω6 (i.e. arachidonic acid, ARA), 22:3ω6, 22:4ω6 or 22:5 ω6.

As non-limiting examples of group c) compounds there may be mentioned flavouring compounds derivable from isopentenyl-PP, as for example menthol.

As non-limiting examples of enzymes of interest there may be mentioned enzymes involved in the carotenoid biosynthesis, as for example those encoded by the genes ispA (farnesyl-diphosphate synthase), crtE (geranylgeranyl diphosphate synthase), crtB (phytoen synthase) and crtl (phytoen desaturase).

The enzymes required for the biosynthesis of lipophilic compounds, in particular those compounds as mentioned above, are well known in the art (see for example: Gerhard Michal, Biochemical Pathways, Spektrum Akademischer Verlag Heidelberg, Berlin (1999); D. Schomburg and D. Stephan, Enzyme Handbook 1-12, Springer Berlin Heidelberg (1996), which are herewith incorporated by reference).

The bacterial cells as used according to the present invention are selected from native or recombinant bacteria having the ability to produce inclusion bodies of the PHA-, TAG- or WE-type, as in particular the TAG-producing nocardioform actinomycetes, in particular of the genus Rhodococcus, Mycobacterium, Nocardia, Gordonia, Skermania and Tsukamurella; as well as TAG-producing Streptomycetes; WE-producing bacteria of the genera Acinetobacter and Alcanivorax; as well as recombinant strains of the genus Escherichia (especially E. coli), Corynebacterium (especially C. glutamicum) and Bacillus (especially B. subtilis). For example the bacterial cells are selected from Rhodococcus opacus PD630 (DSM 44193) and Mycobacterium smegmatis mc²155 (ATCC 700084).

According to a further embodiment of said targeting method bacterial cells are transformed with an expression construct comprising a coding sequence for said fusion protein under the control of a promoter sequence operable in said bacterial host cells.

A further aspect of the invention relates to a method the microbial production of a lipophilic compound of interest, which method comprises cultivating a recombinant bacterial host comprising intracellular inclusion bodies having at least one enzyme which is involved in the biosynthesis of said lipophilic compound targeted in the above manner to said inclusion bodies and cultivating said host under conditions supporting the production of said lipophilic compound.

Preferably said inclusion bodies carrying said lipophilic compound of interest are isolated and said lipophilic compound of interest is recovered from said inclusion bodies. Said lipophilic compound is preferably selected from

a) lipophilic vitamins, derivatives and precursors thereof,
b) fatty acids and fatty alcohols as defined above or
c) flavouring substances.

A further aspect of the invention relates to fusion proteins useful for targeting a protein of interest to an intracellular hydrophobic inclusion body of a bacterial cell, which fusion protein comprises a targeting peptide operatively linked with said protein of interest. Said fusion protein targets the protein of interest in particular to inclusion bodies of the TAG-, WE- or PHA-type, preferably to the TAG-inclusion bodies. Said targeting peptide is preferably as defined above.

In preferred embodiments, the fusion proteins comprise a targeting molecule selected from:

### a) PhaP1 (SEQ ID NO:19)

### b) Perilipin A (SEQ ID NO:27)

### c) ADRP (SEQ ID NO:35)

### d) TIP47 (SEQ ID NO:31)

or a functional equivalent thereof.

In said fusion protein said protein of interest is preferably an enzyme as defined above.

Further aspects of the invention relate to nucleotide sequences encoding a fusion protein of the invention; expression vectors comprising under the control of at least one regulatory sequence a coding sequence for at least one fusion protein as herein defined; recombinant bacterial host cell lines, carrying an expression vector as defined above. Preferably said recombinant bacterial host cell line is derived from a microorganism as defined above.

### 2. Explanation of General Terms

The term “oil bodies”, “lipid bodies” or “inclusion bodies” are herein used synonymously and have to be understood in their broadest sense, comprising those of the TAG-, WE- and PHA-type as described above. Said terms encompass any intracellular structure, which is used by an organism for the purpose of storing energy, carbon or compound required for the biosynthesis of lipophilic products. Said term as used herein includes any or all of the triacylglyceride, phospholipid, wax ester, PHA or protein components present in the complete structure.

As a result of their composition and structure, said bodies may be simply and rapidly separated from liquids of different densities in which they are suspended. For example, in aqueous media where the density is greater than that of the oil bodies, they will float under the influence of gravity or applied centrifugal force. Oil bodies may also be separated from liquids and other solids present in solutions or suspensions by methods that fractionate on the basis of size, for example by using a membrane filter with a pore size less than their diameter.

The term “targeting peptide” encompasses any protein associated with any of the above mentioned intracellular organelles or any functional, i.e. targeting fragment thereof.

### 3. Other Embodiments of the Invention

### 3.1 Proteins According to the Invention

The present invention is not limited to the specifically disclosed “targeting peptides” or “proteins of interest” or fusion proteins thereof, but also extends to functional equivalents thereof.

“Functional equivalents” or analogs of the concretely disclosed enzymes are, within the scope of the present invention, various polypeptides thereof, which moreover possess the desired biological function or activity, e.g. targeting function or enzyme activity.

For example, “functional equivalents” means enzymes which, in a test used for enzymatic activity, display at least a 20%, preferably 50%, especially preferably 75%, quite especially preferably 90% higher or lower activity of an enzyme, as defined herein.

“Functional equivalents” of targeting polypeptides are those, which target to an inclusion body with higher or lower efficiency if compared to a specific example of a targeting polypeptide mentioned herein. For example, the efficiency of a targeting molecule can be analyzed by immunological or enzymatical methods as herein defined and illustrated in the experimental part.

“Functional equivalents”, according to the invention, also means in particular mutants, which, in at least one sequence position of the amino acid sequences stated above, have an amino acid that is different from that concretely stated, but nevertheless possess one of the aforementioned biological activities. “Functional equivalents” thus comprise the mutants obtainable by one or more amino acid additions, substitutions, deletions and/or inversions, where the stated changes can occur in any sequence position, provided they lead to a mutant with the profile of properties according to the invention. Functional equivalence is in particular also provided if the reactivity patterns coincide qualitatively between the mutant and the unchanged polypeptide, i.e. if for example the same substrates are converted at a different rate. Examples of suitable amino acid substitutions are shown in the following table:

| Original residue | Examples of substitution |
|---|---|
| Ala | Ser |
| Arg | Lys |
| Asn | Gln; His |
| Asp | Glu |
| Cys | Ser |
| Gln | Asn |
| Glu | Asp |
| Gly | Pro |
| His | Asn; Gln |
| Ile | Leu; Val |
| Leu | Ile; Val |
| Lys | Arg; Gln; Glu |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

“Functional equivalents” in the above sense are also “precursors” of the polypeptides described, as well as “functional derivatives” and “salts” of the polypeptides.

“Precursors” are in that case natural or synthetic precursors of the polypeptides with or without the desired biological activity.

The expression “salts” means salts of carboxyl groups as well as salts of acid addition of amino groups of the protein molecules according to the invention. Salts of carboxyl groups can be produced in a known way and comprise inorganic salts, for example sodium, calcium, ammonium, iron and zinc salts, and salts with organic bases, for example amines, such as triethanolamine, arginine, lysine, piperidine and the like. Salts of acid addition, for example salts with inorganic acids, such as hydrochloric acid or sulfuric acid and salts with organic acids, such as acetic acid and oxalic acid, are also covered by the invention.

“Functional derivatives” of polypeptides according to the invention can also be produced on functional amino acid side groups or at their N-terminal or C-terminal end using known techniques. Such derivatives comprise for example aliphatic esters of carboxylic acid groups, amides of carboxylic acid groups, obtainable by reaction with ammonia or with a primary or secondary amine; N-acyl derivatives of free amino groups, produced by reaction with acyl groups; or O-acyl derivatives of free hydroxy groups, produced by reaction with acyl groups.

“Functional equivalents” naturally also comprise polypeptides that can be obtained from other organisms, as well as naturally occurring variants. For example, areas of homologous sequence regions can be established by sequence comparison, and equivalent enzymes can be determined on the basis of the concrete parameters of the invention.

“Functional equivalents” also comprise fragments, preferably individual domains or sequence motifs, of the polypeptides according to the invention, which for example display the desired biological function.

“Functional equivalents” are, moreover, fusion proteins, which have one of the polypeptide sequences stated above or functional equivalents derived therefrom and at least one further, functionally different, heterologous sequence in functional N-terminal or C-terminal association (i.e. without substantial mutual functional impairment of the fusion protein parts). Non-limiting examples of these heterologous sequences are e.g. signal peptides, histidine anchors or enzymes.

“Functional equivalents” that are also included according to the invention are homologues of the concretely disclosed proteins. These possess at least 60%, preferably at least 75% in particular at least 85%, e.g. 90, 91, 92, 93, 94, 95, 96, 97, 98 or 99%, homology with the concretely disclosed amino acid sequences, calculated according to the algorithm of Pearson and Lipman, Proc. Natl. Acad, Sci. (USA) 85 (8), 1988, 2444-2448. A percentage homology of a homologous polypeptide according to the invention means in particular the percentage identity of the amino acid residues relative to the total length of one of the amino acid sequences concretely described herein.

In the case of a possible protein glycosylation, “functional equivalents” according to the invention comprise proteins of the type designated above in deglycosylated or glycosylated form as well as modified forms that can be obtained by altering the glycosylation pattern.

Homologues of the proteins or polypeptides according to the invention can be produced by mutagenesis, e.g. by point mutation, lengthening or shortening of the protein.

Homologues of the proteins according to the invention can be identified by screening combinatorial databases of mutants, for example shortening mutants. For example, a variegated database of protein variants can be produced by combinatorial mutagenesis at the nucleic acid level, e.g. by enzymatic ligation of a mixture of synthetic oligonucleotides. There are a great many methods that can be used for the production of databases of potential homologues from a degenerated oligonucleotide sequence. Chemical synthesis of a degenerated gene sequence can be carried out in an automatic DNA synthesizer, and the synthetic gene can then be ligated in a suitable expression vector. The use of a degenerated genome makes it possible to supply all sequences in a mixture, which code for the desired set of potential protein sequences. Methods of synthesis of degenerated oligonucleotides are known to a person skilled in the art (e.g. Narang, S. A. (1983) Tetrahedron 39:3; Itakura et al. (1984) Annu. Rev. Biochem. 53:323; Itakura et al., (1984) Science 198:1056; Ike et al. (1983) Nucleic Acids Res. 11:477).

In the prior art, several techniques are known for the screening of gene products of combinatorial databases, which were produced by point mutations or shortening, and for the screening of cDNA libraries for gene products with a selected property. These techniques can be adapted for the rapid screening of the gene banks that were produced by combinatorial mutagenesis of homologues according to the invention. The techniques most frequently used for the screening of large gene banks, which are based on a high-throughput analysis, comprise cloning of the gene bank in expression vectors that can be replicated, transformation of the suitable cells with the resultant vector database and expression of the combinatorial genes in conditions in which detection of the desired activity facilitates isolation of the vector that codes for the gene whose product was detected. Recursive Ensemble Mutagenesis (REM), a technique that increases the frequency of functional mutants in the databases, can be used in combination with the screening tests, in order to identify homologues (Arkin and Yourvan (1992) PNAS 89:7811-7815; Delgrave et al. (1993) Protein Engineering 6 (3):327-331).

### 3.2 Coding Nucleic Acid Sequences

The invention also relates to nucleic acid sequences that code for fusion proteins as defined herein.

The present invention also relates to nucleic acids with a certain degree of “identity” to the sequences specifically disclosed herein. “Identity” between two nucleic acids means identity of the nucleotides, in each case over the entire length of the nucleic acid, in particular the identity calculated by means of the Vector NTI Suite 7.1 program of the company Informax (USA) employing the Clustal Method (Higgins D G, Sharp P M. Fast and sensitive multiple sequence alignments on a microcomputer. Comput Appl. Biosci. 1989 April; 5 (2):151-1) with the following settings:

### Multiple Alignment Parameter:

| | |
|---|---|
| Gap opening penalty | 10 |
| Gap extension penalty | 10 |
| Gap separation penalty range | 8 |
| Gap separation penalty | off |
| % identity for alignment delay | 40 |
| Residue specific gaps | off |
| Hydrophilic residue gap | off |
| Transition weighing | 0 |

### Pairwise Alignment Parameter:

| FAST algorithm | on |
|---|---|
| K-tuple size | 1 |
| Gap penalty | 3 |
| Window size | 5 |
| Number of best diagonals | 5 |

All the nucleic acid sequences mentioned herein (single-stranded and double-stranded DNA and RNA sequences, for example cDNA and mRNA) can be produced in a known way by chemical synthesis from the nucleotide building blocks, e.g. by fragment condensation of individual overlapping, complementary nucleic acid building blocks of the double helix. Chemical synthesis of oligonucleotides can, for example, be performed in a known way, by the phosphoamidite method (Voet, Voet, 2nd edition, Wiley Press, New York, pages 896-897). The accumulation of synthetic oligonucleotides and filling of gaps by means of the Klenow fragment of DNA polymerase and ligation reactions as well as general cloning techniques are described in Sambrook et al. (1989), see below.

The invention also relates to nucleic acid sequences (single-stranded and double-stranded DNA and RNA sequences, e.g. cDNA and mRNA), coding for one of the above polypeptides and their functional equivalents, which can be obtained for example using artificial nucleotide analogs.

The invention relates both to isolated nucleic acid molecules, which code for polypeptides or proteins according to the invention or biologically active segments thereof, and to nucleic acid fragments, which can be used for example as hybridization probes or primers for identifying or amplifying coding nucleic acids according to the invention.

The nucleic acid molecules according to the invention can in addition contain untranslated sequences from the 3′ and/or 5′ end of the coding genetic region.

The invention further relates to the nucleic acid molecules that are complementary to the concretely described nucleotide sequences or a segment thereof.

The nucleotide sequences according to the invention make possible the production of probes and primers that can be used for the identification and/or cloning of homologous sequences in other cellular types and organisms. Such probes or primers generally comprise a nucleotide sequence region which hybridizes under “stringent” conditions (see below) on at least about 12, preferably at least about 25, for example about 40, 50 or 75 successive nucleotides of a sense strand of a nucleic acid sequence according to the invention or of a corresponding antisense strand.

An “isolated” nucleic acid molecule is separated from other nucleic acid molecules that are present in the natural source of the nucleic acid and can moreover be substantially free from other cellular material or culture medium, if it is being produced by recombinant techniques, or can be free from chemical precursors or other chemicals, if it is being synthesized chemically.

A nucleic acid molecule according to the invention can be isolated by means of standard techniques of molecular biology and the sequence information supplied according to the invention. For example, cDNA can be isolated from a suitable cDNA library, using one of the concretely disclosed complete sequences or a segment thereof as hybridization probe and standard hybridization techniques (as described for example in Sambrook, J., Fritsch, E. F. and Maniatis, T. Molecular Cloning: A Laboratory Manual. 2nd edition, Cold Spring Harbor Laboratory, Cold Spring Harbor Laboratory Press, Cold Spring Harbor, N.Y., 1989). In addition, a nucleic acid molecule comprising one of the disclosed sequences or a segment thereof, can be isolated by the polymerase chain reaction, using the oligonucleotide primers that were constructed on the basis of this sequence. The nucleic acid amplified in this way can be cloned in a suitable vector and can be characterized by DNA sequencing. The oligonucleotides according to the invention can also be produced by standard methods of synthesis, e.g. using an automatic DNA synthesizer.

Nucleic acid sequences according to the invention or derivatives thereof, homologues or parts of these sequences, can for example be isolated by usual hybridization techniques or the PCR technique from other bacteria, e.g. via genomic or cDNA libraries. These DNA sequences hybridize in standard conditions with the sequences according to the invention.

“Hybridize” means the ability of a polynucleotide or oligonucleotide to bind to an almost complementary sequence in standard conditions, whereas nonspecific binding does not occur between non-complementary partners in these conditions. For this, the sequences can be 90-100% complementary. The property of complementary sequences of being able to bind specifically to one another is utilized for example in Northern Blotting or Southern Blotting or in primer binding in PCR or RT-PCR.

Short oligonucleotides of the conserved regions are used advantageously for hybridization. However, it is also possible to use longer fragments of the nucleic acids according to the invention or the complete sequences for the hybridization. These standard conditions vary depending on the nucleic acid used (oligonucleotide, longer fragment or complete sequence) or depending on which type of nucleic acid—DNA or RNA—is used for hybridization. For example, the melting temperatures for DNA:DNA hybrids are approx. 10° C. lower than those of DNA:RNA hybrids of the same length.

For example, depending on the particular nucleic acid, standard conditions mean temperatures between 42 and 58° C. in an aqueous buffer solution with a concentration between 0.1 to 5×SSC (1×SSC=0.15 M NaCl, 15 mM sodium citrate, pH 7.2) or additionally in the presence of 50% formamide, for example 42° C. in 5×SSC, 50% formamide. Advantageously, the hybridization conditions for DNA:DNA hybrids are 0.1×SSC and temperatures between about 20° C. to 45° C., preferably between about 30° C. to 45° C. For DNA:RNA hybrids the hybridization conditions are advantageously 0.1×SSC and temperatures between about 30° C. to 55° C., preferably between about 45° C. to 55° C. These stated temperatures for hybridization are examples of calculated melting temperature values for a nucleic acid with a length of approx. 100 nucleotides and a G+C content of 50% in the absence of formamide. The experimental conditions for DNA hybridization are described in relevant genetics textbooks, for example Sambrook et al., 1989, and can be calculated using formulae that are known by a person skilled in the art, for example depending on the length of the nucleic acids, the type of hybrids or the G+C content. A person skilled in the art can obtain further information on hybridization from the following textbooks: Ausubel et al. (eds), 1985, Current Protocols in Molecular Biology, John Wiley & Sons, New York; Hames and Higgins (eds), 1985, Nucleic Acids Hybridization: A Practical Approach, IRL Press at Oxford University Press, Oxford; Brown (ed), 1991, Essential Molecular Biology: A Practical Approach, IRL Press at Oxford University Press, Oxford.

“Hybridization” can in particular be carried out under stringent conditions. Such hybridization conditions are for example described in Sambrook, J., Fritsch, E. F., Maniatis, T., in: Molecular Cloning (A Laboratory Manual), 2nd edition, Cold Spring Harbor Laboratory Press, 1989, pages 9.31-9.57 or in Current Protocols in Molecular Biology, John Wiley & Sons, N.Y. (1989), 6.3.1-6.3.6.

“Stringent” hybridization conditions mean in particular: Incubation at 42° C. overnight in a solution consisting of 50% formamide, 5×SSC (750 mM NaCl, 75 mM tri-sodium citrate), 50 mM sodium phosphate (pH 7.6), 5×Denhardt Solution, 10% dextran sulfate and 20 g/ml denatured, sheared salmon sperm DNA, followed by washing of the filters with 0.1×SSC at 65° C.

The invention also relates to derivatives of the concretely disclosed or derivable nucleic acid sequences.

Thus, further nucleic acid sequences according to the invention can be derived from the sequences specifically disclosed herein and can differ from it by addition, substitution, insertion or deletion of individual or several nucleotides, and furthermore code for polypeptides with the desired profile of properties.

The invention also encompasses nucleic acid sequences that comprise so-called silent mutations or have been altered, in comparison with a concretely stated sequence, according to the codon usage of a special original or host organism, as well as naturally occurring variants, e.g. splicing variants or allelic variants, thereof.

It also relates to sequences that can be obtained by conservative nucleotide substitutions (i.e. the amino acid in question is replaced by an amino acid of the same charge, size, polarity and/or solubility).

The invention also relates to the molecules derived from the concretely disclosed nucleic acids by sequence polymorphisms. These genetic polymorphisms can exist between individuals within a population owing to natural variation. These natural variations usually produce a variance of 1 to 5% in the nucleotide sequence of a gene.

Derivatives of nucleic acid sequences according to the invention mean for example allelic variants, having at least 60% homology at the level of the derived amino acid, preferably at least 80% homology, quite especially preferably at least 90% homology over the entire sequence range (regarding homology at the amino acid level, reference should be made to the details given above for the polypeptides). Advantageously, the homologies can be higher over partial regions of the sequences.

Furthermore, derivatives are also to be understood to be homologues of the nucleic acid sequences according to the invention, for example animal, plant, fungal or bacterial homologues, shortened sequences, single-stranded DNA or RNA of the coding and noncoding DNA sequence. For example, homologues have, at the DNA level, a homology of at least 40%, preferably of at least 60%, especially preferably of at least 70%, quite especially preferably of at least 80% over the entire DNA region given in a sequence specifically disclosed herein.

Moreover, derivatives are to be understood to be, for example, fusions with promoters. The promoters that are added to the stated nucleotide sequences can be modified by at least one nucleotide exchange, at least one insertion, inversion and/or deletion, though without impairing the functionality or efficacy of the promoters. Moreover, the efficacy of the promoters can be increased by altering their sequence or can be exchanged completely with more effective promoters even of organisms of a different genus.

### 3.3 Constructs According to the Invention

The invention also relates to expression constructs, containing, under the genetic control of regulatory nucleic acid sequences, a nucleic acid sequence coding for a polypeptide or fusion protein according to the invention; as well as vectors comprising at least one of these expression constructs.

“Expression unit” means, according to the invention, a nucleic acid with expression activity, which comprises a promoter as defined herein and, after functional association with a nucleic acid that is to be expressed or a gene, regulates the expression, i.e. the transcription and the translation of this nucleic acid or of this gene. In this context, therefore, it is also called a “regulatory nucleic acid sequence”. In addition to the promoter, other regulatory elements may be present, e.g. enhancers.

“Expression cassette” or “expression construct” means, according to the invention, an expression unit, which is functionally associated with the nucleic acid that is to be expressed or the gene that is to be expressed. In contrast to an expression unit, an expression cassette thus comprises not only nucleic acid sequences which regulate transcription and translation, but also the nucleic acid sequences which should be expressed as protein as a result of the transcription and translation.

The terms “expression” or “overexpression” describe, in the context of the invention, the production or increase of intracellular activity of one or more enzymes in a microorganism, which are encoded by the corresponding DNA. For this, it is possible for example to insert a gene in an organism, replace an existing gene by another gene, increase the number of copies of the gene or genes, use a strong promoter or use a gene that codes for a corresponding enzyme with a high activity, and optionally these measures can be combined.

Preferably such constructs according to the invention comprise a promoter 5′-upstream from the respective coding sequence, and a terminator sequence 3′-downstream, and optionally further usual regulatory elements, in each case functionally associated with the coding sequence.

A “promotor”, a “nucleic acid with promotor activity” or a “promotor sequence” mean, according to the invention, a nucleic acid which, functionally associated with a nucleic acid that is to be transcribed, regulates the transcription of this nucleic acid.

“Functional” or “operative” association means, in this context, for example the sequential arrangement of one of the nucleic acids with promoter activity and of a nucleic acid sequence that is to be transcribed and optionally further regulatory elements, for example nucleic acid sequences that enable the transcription of nucleic acids, and for example a terminator, in such a way that each of the regulatory elements can fulfill its function in the transcription of the nucleic acid sequence. This does not necessarily require a direct association in the chemical sense. Genetic control sequences, such as enhancer sequences, can also exert their function on the target sequence from more remote positions or even from other DNA molecules. Arrangements are preferred in which the nucleic acid sequence that is to be transcribed is positioned behind (i.e. at the 3′ end) the promoter sequence, so that the two sequences are bound covalently to one another. The distance between the promoter sequence and the nucleic acid sequence that is to be expressed transgenically can be less than 200 bp (base pairs), or less than 100 bp or less than 50 bp.

Apart from promoters and terminators, examples of other regulatory elements that may be mentioned are targeting sequences, enhancers, polyadenylation signals, selectable markers, amplification signals, replication origins and the like. Suitable regulatory sequences are described for example in Goeddel, Gene Expression Technology: Methods in Enzymology 185, Academic Press, San Diego, Calif. (1990).

Nucleic acid constructs according to the invention comprise in particular sequences selected from those, specifically mentioned herein or derivatives and homologues thereof, as well as the nucleic acid sequences that can be derived from amino acid sequences specifically mentioned herein which are advantageously associated operatively or functionally with one or more regulating signal for controlling, e.g. increasing, gene expression.

In addition to these regulatory sequences, the natural regulation of these sequences can still be present in front of the actual structural genes and optionally can have been altered genetically, so that natural regulation is switched off and the expression of the genes has been increased. The nucleic acid construct can also be of a simpler design, i.e. without any additional regulatory signals being inserted in front of the coding sequence and without removing the natural promoter with its regulation. Instead, the natural regulatory sequence is silenced so that regulation no longer takes place and gene expression is increased.

A preferred nucleic acid construct advantageously also contains one or more of the aforementioned enhancer sequences, functionally associated with the promoter, which permit increased expression of the nucleic acid sequence. Additional advantageous sequences, such as other regulatory elements or terminators, can also be inserted at the 3′ end of the DNA sequences. One or more copies of the nucleic acids according to the invention can be contained in the construct. The construct can also contain other markers, such as antibiotic resistances or auxotrophy-complementing genes, optionally for selection on the construct.

Examples of suitable regulatory sequences are contained in promoters such as cos-, tac-, trp-, tet-, trp-tet-, lpp-, lac-, lpp-lac-, lacI^{q-}, T7-, T5-, T3-, gal-, trc-, ara-, rhaP (rhaP_{BAD})SP6-, lambda-P_{R}- or in the lambda-P_{L} promoter, which find application advantageously in Gram-negative bacteria. Other advantageous regulatory sequences are contained for example in the Gram-positive promoters ace, amy and SPO2, in the yeast or fungal promoters ADC1, MFalpha, AC, P-60, CYC1, GAPDH, TEF, rp28, ADH. Artificial promoters can also be used for regulation.

For expression, the nucleic acid construct is inserted in a host organism advantageously in a vector, for example a plasmid or a phage which permits optimum expression of the genes in the host. In addition to plasmids and phages, vectors are also to be understood as meaning all other vectors known to a person skilled in the art, e.g. viruses, such as SV40, CMV, baculovirus and adenovirus, transposons, IS elements, phasmids, cosmids, and linear or circular DNA. These vectors can be replicated autonomously in the host organism or can be replicated chromosomally. These vectors represent a further embodiment of the invention.

Suitable plasmids are, for example in E. coli, pLG338, pACYC184, pBR322, pUC18, pUC19, pKC30, pRep4, pHS1, pKK223-3, pDHE19.2, pHS2, pPLc236, pMBL24, pLG200, pUR290, pIN-III¹¹³-B1, λgt11 or pBdCl; in nocardioform actinomycetes pJAM2; in Streptomyces pIJ101, pIJ364, pIJ702 or pIJ361; in bacillus pUB110, pC194 or pBD214; in Corynebacterium pSA77 or pAJ667; in fungi pALS1, pIL2 or pBB116; in yeasts 2alphaM, pAG-1, YEp6, YEp13 or pEMBLYe23 or in plants pLGV23, pGHIac⁺, pBIN19, pAK2004 or pDH51. The aforementioned plasmids represent a small selection of the possible plasmids. Other plasmids are well known to a person skilled in the art and will be found for example in the book Cloning Vectors (Eds. Pouwels P. H. et al. Elsevier, Amsterdam-New York-Oxford, 1985, ISBN 0 444 904018).

In a further embodiment of the vector, the vector containing the nucleic acid construct according to the invention or the nucleic acid according to the invention can be inserted advantageously in the form of a linear DNA in the microorganisms and integrated into the genome of the host organism through heterologous or homologous recombination. This linear DNA can comprise a linearized vector such as plasmid or just the nucleic acid construct or the nucleic acid according to the invention.

For optimum expression of heterologous genes in organisms, it is advantageous to alter the nucleic acid sequences in accordance with the specific codon usage employed in the organism. The codon usage can easily be determined on the basis of computer evaluations of other, known genes of the organism in question.

The production of an expression cassette according to the invention is based on fusion of a suitable promoter with a suitable coding nucleotide sequence and a terminator signal or polyadenylation signal. Common recombination and cloning techniques are used for this, as described for example in T. Maniatis, E. F. Fritsch and J. Sambrook, Molecular Cloning: A Laboratory Manual, Cold Spring Harbor Laboratory, Cold Spring Harbor, N.Y. (1989) as well as in T. J. Silhavy, M. L. Berman and L. W. Enquist, Experiments with Gene Fusions, Cold Spring Harbor Laboratory, Cold Spring Harbor, N.Y. (1984) and in Ausubel, F. M. et al., Current Protocols in Molecular Biology, Greene Publishing Assoc. and Wiley Interscience (1987).

The recombinant nucleic acid construct or gene construct is inserted advantageously in a host-specific vector for expression in a suitable host organism, to permit optimum expression of the genes in the host. Vectors are well known to a person skilled in the art and will be found for example in “Cloning Vectors” (Pouwels P. H. et al., Publ. Elsevier, Amsterdam-New York-Oxford, 1985).

3.4 Hosts that can be Used According to the Invention

Depending on the context, the term “microorganism” means the starting microorganism (wild-type) or a genetically modified microorganism according to the invention, or both.

The term “wild-type” means, according to the invention, the corresponding starting microorganism, and need not necessarily correspond to a naturally occurring organism.

By means of the vectors according to the invention, recombinant microorganisms can be produced, which have been transformed for example with at least one vector according to the invention and can be used for production of the polypeptides according to the invention. Advantageously, the recombinant constructs according to the invention, described above, are inserted in a suitable host system and expressed. Preferably, common cloning and transfection methods that are familiar to a person skilled in the art are used, for example co-precipitation, protoplast fusion, electroporation, retroviral transfection and the like, in order to secure expression of the stated nucleic acids in the respective expression system. Suitable systems are described for example in Current Protocols in Molecular Biology, F. Ausubel et al., Publ. Wiley Interscience, New York 1997, or Sambrook et al. Molecular Cloning: A Laboratory Manual. 2nd edition, Cold Spring Harbor Laboratory, Cold Spring Harbor Laboratory Press, Cold Spring Harbor, N.Y., 1989.

In principle, all prokaryotic organisms can be considered as recombinant host organisms for the nucleic acid according to the invention or the nucleic acid construct. Bacteria are used advantageously as host organisms. Preferably they are selected from native or recombinant bacteria having the ability to produce inclusion bodies of the PHA-, TAG- or WE-type, as in particular the TAG-producing nocardioform actinomycetes, in particular of the genus Rhodococcus, Mycobacterium, Nocardia, Gordonia, Skermania and Tsukamurella; as well as TAG-producing Streptomycetes; WE-producing genera Acinetobacter and Alcanivorax, as well as recombinant strains of the genus Escherichia, especially E. coli, Corynebacterium, especially C. glutamicum and Bacillus, especially B. subtilis.

The host organism or host organisms according to the invention then preferably contain at least one of the nucleic acid sequences, nucleic acid constructs or vectors described in this invention, which code for an enzyme activity according to the above definition.

The organisms used in the method according to the invention are grown or bred in a manner familiar to a person skilled in the art, depending on the host organism. As a rule, microorganisms are grown in a liquid medium, which contains a source of carbon, generally in the form of sugars, a source of nitrogen generally in the form of organic sources of nitrogen such as yeast extract or salts such as ammonium sulfate, trace elements such as iron, manganese and magnesium salts and optionally vitamins, at temperatures between 0° C. and 100° C., preferably between 10° C. to 60° C. with oxygen aeration. The pH of the liquid nutrient medium can be maintained at a fixed value, i.e. regulated or not regulated during growing. Growing can be carried out batchwise, semi-batchwise or continuously. Nutrients can be supplied at the start of fermentation or can be supplied subsequently, either semi-continuously or continuously.

### 3.5 Recombinant Production of the Lipophilic Compounds

The invention also relates to methods for production of lipophilic compounds according to the invention by cultivating a fusion protein producing microorganism which expresses a fusion protein of the invention, wherein cultivation is performed under conditions allowing the enzymatic production of said lipophilic compound, and isolating the desired compound from the culture. The compounds can also be produced on an industrial scale in this way, if so desired.

Said microorganism may express one or more fusions proteins providing the required enzyme activity or activities for the synthesis of the desired lipophilic compound. Due to the close proximity of enzyme activity and lipid body it is expected that the produced lipophilic product will associate with, i.e. be incorporated into and/or adsorbed to, the lipid body. This will shift the equilibrium of the enzymatic reaction further in the direction of the desired product. Moreover, the product associated with the lipid body can more easily be separated from the bulk of the biomass and purified by means of conventional purification methods, as for example extraction and chromatography.

Prior to initiating the biosynthesis of the desired lipophilic product it is of course of advantage to take care that sufficient lipid body carrier is provided within the bacterial cell. This can conveniently be achieved by cultivating the cells under so-called storage conditions, as for example illustrated for specific strains in the attached examples. Afterwards of simultaneously the recombinant expression of the required fusion proteins is induced so that sufficient enzymatic activity is targeted to the lipid bodies. In cases where the bacterial cells are unable to produce (at all or in sufficient amount) the substrate(s) and/or co-substrate(s) required for the enzymatic production of the desired lipophilic end product the required educts can be added to the culture medium.

The microorganisms as used according to the invention can be cultivated continuously or discontinuously in the batch process or in the fed batch or repeated fed batch process. A review of known methods of cultivation will be found in the textbook by Chmiel (Bioprocesstechnik 1. Einführung in die Bioverfahrenstechnik (Gustav Fischer Verlag, Stuttgart, 1991)) or in the textbook by Storhas (Bioreaktoren und periphere Einrichtungen (Vieweg Verlag, Braunschweig/Wiesbaden, 1994)).

The culture medium that is to be used must satisfy the requirements of the particular strains in an appropriate manner. Descriptions of culture media for various microorganisms are given in the handbook “Manual of Methods for General Bacteriology” of the American Society for Bacteriology (Washington D.C., USA, 1981).

These media that can be used according to the invention generally comprise one or more sources of carbon, sources of nitrogen, inorganic salts, vitamins and/or trace elements.

Preferred sources of carbon are sugars, such as mono-, di- or polysaccharides. Very good sources of carbon are for example glucose, fructose, mannose, galactose, ribose, sorbose, ribulose, lactose, maltose, sucrose, raffinose, starch or cellulose. Sugars can also be added to the media via complex compounds, such as molasses, or other by-products from sugar refining.

It may also be advantageous to add mixtures of various sources of carbon. Other possible sources of carbon are oils and fats such as soybean oil, sunflower oil, peanut oil and coconut oil, fatty acids such as palmitic acid, stearic acid or linoleic acid, alcohols such as glycerol, methanol or ethanol and organic acids such as acetic acid or lactic acid.

Sources of nitrogen are usually organic or inorganic nitrogen compounds or materials containing these compounds. Examples of sources of nitrogen include ammonia gas or ammonium salts, such as ammonium sulfate, ammonium chloride, ammonium phosphate, ammonium carbonate or ammonium nitrate, nitrates, urea, amino acids or complex sources of nitrogen, such as corn-steep liquor, soybean flour, soybean protein, yeast extract, meat extract and others. The sources of nitrogen can be used separately or as a mixture.

Inorganic salt compounds that may be present in the media comprise the chloride, phosphate or sulfate salts of calcium, magnesium, sodium, cobalt, molybdenum, potassium, manganese, zinc, copper and iron.

Inorganic sulfur-containing compounds, for example sulfates, sulfites, dithionites, tetrathionates, thiosulfates, sulfides, but also organic sulfur compounds, such as mercaptans and thiols, can be used as sources of sulfur.

Phosphoric acid, potassium dihydrogenphosphate or dipotassium hydrogenphosphate or the corresponding sodium-containing salts can be used as sources of phosphorus.

Chelating agents can be added to the medium, in order to keep the metal ions in solution. Especially suitable chelating agents comprise dihydroxyphenols, such as catechol or protocatechuate, or organic acids, such as citric acid.

The fermentation media used according to the invention usually also contain other growth factors, such as vitamins or growth promoters, which include for example biotin, riboflavin, thiamine, folic acid, nicotinic acid, pantothenate and pyridoxine. Growth factors and salts often come from complex components of the media, such as yeast extract, molasses, corn-steep liquor and the like. In addition, suitable precursors can be added to the culture medium. The precise composition of the compounds in the medium is strongly dependent on the particular experiment and must be decided individually for each specific case. Information on media optimization can be found in the textbook “Applied Microbiol. Physiology, A Practical Approach” (Publ. P. M. Rhodes, P. F. Stanbury, IRL Press (1997) p. 53-73, ISBN 0 19 963577 3). Growing media can also be obtained from commercial suppliers, such as Standard 1 (Merck) or BHI (Brain heart infusion, DIFCO) etc.

All components of the medium are sterilized, either by heating (20 min at 1.5 bar and 121° C.) or by sterile filtration. The components can be sterilized either together, or if necessary separately. All the components of the medium can be present at the start of growing, or optionally can be added continuously or by batch feed.

The temperature of the culture is normally between 15° C. and 45° C., preferably 25° C. to 40° C. and can be kept constant or can be varied during the experiment. The pH value of the medium should be in the range from 5 to 8.5, preferably around 7.0. The pH value for growing can be controlled during growing by adding basic compounds such as sodium hydroxide, potassium hydroxide, ammonia or ammonia water or acid compounds such as phosphoric acid or sulfuric acid. Antifoaming agents, e.g. fatty acid polyglycol esters, can be used for controlling foaming. To maintain the stability of plasmids, suitable substances with selective action, e.g. antibiotics, can be added to the medium. Oxygen or oxygen-containing gas mixtures, e.g. the ambient air, are fed into the culture in order to maintain aerobic conditions. The temperature of the culture is normally from 20° C. to 45° C. Culture is continued until a maximum of the desired product has formed. This is normally achieved within 10 hours to 160 hours.

The cells can be disrupted optionally by high-frequency ultrasound, by high pressure, e.g. in a French pressure cell, by osmolysis, by the action of detergents, lytic enzymes or organic solvents, by means of homogenizers or by a combination of several of the methods listed.

The following examples only serve to illustrate the invention. The numerous possible variations that are obvious to a person skilled in the art also fall within the scope of the invention.

### EXPERIMENTAL PART

### 1. Materials and Methods

### a) Strains, Plasmids and Culture Conditions

Cells of Escherichia coli strains XL1 blue (Stratagene) and S17-1 (Simon et al. [22a]) were routinely cultivated in Luria-Bertani (LB) medium (Sambrook et al. [21]). Cells of Rhodococcus opacus PD630 (DSM 44193, Alvarez et al. [2]) and Mycobacterium smegmatis mc²155 (ATCC 700084, Snapper et al. [23]) were cultivated in Standard I (StdI) medium (Merck).

To promote biosynthesis of TAGs and formation of inclusions, cells were transferred to mineral salt medium (MSM) containing 0.1 g l⁻¹ NH₄Cl and cultivated for 24, 48 and 72 h (Schlegel et al., [22]). In addition, M. smegmatis mc²155 was also cultivated in Sauton's medium (SM) (Darzins, 1958). To promote TAG accumulation in SM, the potassium phosphate concentration was reduced to 0.05 g l⁻¹. Carbon was supplied in MSM and SM as sodium gluconate or glucose (10 g l⁻¹) for R. opacus PD630 or M. smegmatis mc²155, respectively. To maintain plasmid pJAM2 and derivatives, kanamycin was used at a final concentration of 50 μg ml⁻¹ according to Sambrook et al. [21]). Induction of the acetamidase (ace) promotor of pJAM2 and derivatives was achieved by addition of 0.5% (w/v) acetamide to the respective cultures (Triccas et al. [29]). All liquid cultures were performed in Erlenmeyer flasks equipped with baffles at 37° C. for E. coli or at 30° C. for R. opacus PD630 and M. smegmatis mc²155, respectively. Solid media were prepared by the addition of 18 g l⁻¹ agar.

### b) Preparations of the Electrocompetent Cells

Plasmids were transferred to R. opacus PD630 and M. smegmatis mc²155 by electroporation in a model 2550 electroporator (Eppendorf-Netheler-Hinz, Hamburg, Germany). Preparation of electrocompetent cells was done as described by Kalscheuer et al. [10] for R. opacus PD630 and by Snapper et al. [23] for M. smegmatis mc²155.

### c) Preparation of Crude Cell Extracts, Soluble Fractions and TAG Inclusions.

Cells of R. opacus and M. smegmatis were grown in MSM with reduced ammonium concentration as described above, harvested by centrifugation (20 min, 6000×g, 4° C.) and resuspended in two volumes of 0.1 M sodium phosphate buffer (pH 7.5). After threefold passage through a French pressure cell (1000 MPa), crude extracts were obtained. To obtain soluble fractions, cell debris was removed from crude extracts by centrifugation for 30 min at 16000×g at 4° C. followed by a 90 min 100000×g centrifugation step at 4° C. in a Sorvall Discovery 90SE ultracentrifuge. Membrane fragments were pelleted by the 100000×g ultracentrifugation step and subsequently resuspended in 0.1 mM sodium phosphate buffer (pH 7.5) after washing in the same buffer. TAG inclusions were prepared by loading 1-2 ml of crude extracts onto the top of a discontinous glycerol gradient. The discontinous glycerol gradient consisted of each 3 ml of 22, 44 and 88% (v/v) glycerol in 0.1 M sodium phosphate buffer (pH 7.5). The gradient was centrifuged for 1 h at 170000×g at 4° C. The TAG inclusions were withdrawn and subsequently washed twice in 0.1 M sodium phosphate buffer (pH 7.5) and used for further analyses.

### d) Determination of β-Galactosidase Activities on Isolated TAG Inclusions.

TAG inclusions isolated from cells of R. opacus PD630 harbouring pJAM2::phaP1-lacZ or pJAM2::phaP1 as a control were prepared as described above. Inclusions (10 mg wet weight) were suspended in 100 μl of 0.1 M sodium phosphate buffer (pH 7.5), followed by addition of 650 μl reaction solution consisting of 17 ml 0.1 M sodium phosphate buffer (pH 7.5), 3 ml ortho-nitrophenyl-β-D-galactopyranoside (ONPG) solution (8%, w/v), 1 mM magnesium chloride, 45 mM β-mercaptoethanol and 4 μl SDS solution (20%, w/v). The assay was incubated for 30 min at 37° C. To stop the reaction, 400 μl of 1 M disodium carbonate were added. Subsequently, TAG inclusions were eliminated from the assay by filtration, and the absorbance of the filtrate was examined at 405 nm to analyze the amount of cleaved ONPG. For calculation of enzyme activities an ε₄₀₅ₙₘ of 4.6 mM⁻¹ cm⁻¹ was used for the respective product ONP (ortho-nitrophenol). Measured β-galactosidase activity was essentially associated with TAG inclusions, since further cleavage of ONPG did not occur in assays after the inclusions were removed.

### e) Immunoblot Analysis.

Known amounts of cell lysates or subcellular fractions based on equivalent protein concentrations were resolved in sodium dodecylsulfate (SDS)-polyacrylamide gels and transferred onto a polyvinylidene (PVDF) membrane according to the method of Towbin et al. [28]. Proteins on the membrane were stained with Ponceau S and analyzed immunologically employing polyclonal chicken anti-maize oleosin IgGs [19], polyclonal rabbit anti-murine PAT IgGs (gift from C. Londos), a polyclonal antibody raised in guinea pig against a synthetic polypeptide representing the N-terminus (amino acids 1-16) of human TIP47 (GP30; Progen Biotechnik) polyclonal rabbit anti-PhaP1 IgG (Wieczorek et al., [31a]) and mouse monoclonal antibody to a synthetic peptide representing the N-terminus (amino acids 5-27) of human ADRP (AP125; Progen Biotechnik), respectively. IgGs were visualized on immunoblots using goat anti-rabbit, anti-murine, or anti-chicken IgGs alkaline phosphatase conjugates, respectively, converting 5-bromo-4-chloro-3-indolyl-phosphate dipotassium/nitrotetrazolium blue chloride into an insoluble and dark product (Sigma).

### f) Microscopy.

Nile Red labeled cells and isolated TAG inclusions were prepared by incubating samples 30 min at 4° C. in 0.1 M sodium phosphate buffer (pH 7.5) containing 0.5 μg ml⁻¹ Nile Red (stock solution 0.5 mg ml⁻¹ in dimethyl sulfoxide). After labelling, cells and inclusions were sedimented by centrifugation at 16000×g at 4° C. and resuspended in 0.1 M sodium phosphate buffer (pH 7.5).

Cells and TAG inclusions were attached to glass slides via electrostatic interaction, which became positively charged through adsorption of poly(α-L-lysine) (PL) hydrobromide. In order to coat a glass surface with PL hydrobromide, cleaned glass slides were rinsed throughoutly with tap water, dipped in methanol, and again rinsed with demineralized water. Afterwards a drop of 0.01% (w/v) PL hydrobromide solution was added. After air-drying, slides were rinsed with demineralized water, and a drop of a cell suspension or TAG inclusions was added. After 15 min, the coated slides were rinsed with demineralized water to remove loosely attached bacteria or TAG inclusions and transferred to fluorescence microscopy.

Slides were examined on a Zeiss Axio Imager M1 upright wide field fluorescence microscope fitted with a 100×/1.4 NA oil-immersion Plan-Apochromat objective lens and 4× or 2.5× auxiliary tube lenses in phase contrast (PH) or differential interference contrast (DIC) mode. Images were collected by using a peltier cooled AxioCam MRm 16 bit digital monochrome charge-coupled device camera (CCD). The ⅔″ sized CCD chip consisted of 1388 (H)×1040 (V) pixels, each 6.45×6.45 nm in size. Nile Red and eGFP fluorescence were excited using a Zeiss HBO 103 W/2 high-pressure mercury arc lamp. Recording of single and multichannel fluorescence images were performed by using emission bandpass filters at EX/EM 470±40/525±50 nm for eGFP and EX/EM 550±25/605±70 nm for Nile Red. Image stacks consisting of 45-96 planes of optical sections covering the entire z-axis were generated by collecting images at focal positions differing in increments of 0.275 μm by employing a high-precision motorized xyz stage. Depending on samples and fluorescence channels, the exposition times varied between 50 and 1000 ms to obtain sufficiently saturated images suitable for deconvolution. To reduce photobleaching, illumination was controlled by a Zeiss high speed shutter device. Care was taken to avoid exposing the field to be recorded to the fluorescence light source until recording had begun and the camera had been adjusted to provide the optimum image. Images were stored in zvi data format for subsequent image data processing. All images were acquired using the Zeiss Axiovision 4.5 software. Where indicated, constrained iterative deconvolution of acquired images was performed using the Zeiss AxioVision 3D deconvolution module. All image processing was performed on a Siemens 2.8 GHz Line Celsius R630 workstation.

### g) Freeze-Fracturing, Cryosectioning and Immunogold Labeling.

For cryosectioning, cell suspensions were prefixed for 5 min by adding an equal volume of 4% (w/v) paraformaldehyde in phosphate buffered saline (PBS) (pH 7.4). Cells were washed briefly in the same buffer and fixed further in 4% (w/v) paraformaldehyde for 1 h followed by incubation in 4% (w/v) paraformaldehyde with 0.9 M sucrose and 90% (w/v) polyvinylpyrrolidone 25 buffered with 50 mM sodium carbonate (pH 7.0) as a cryoprotectant for 1 h. The cells were concentrated by centrifugation, placed on pins in a small volume of cryoprotectant, and frozen in liquid nitrogen. Ultrathin sections were performed as described by Tokuyasu [17]. For freeze-fracturing, cell suspensions (700 ml) were pelleted by centrifugation for 30 min at 6,000×g and 4° C., resuspended in 30% (v/v) glycerol (<30 sec), fixed in Freon 22 cooled with liquid nitrogen, and freeze fractured in a BA310 freeze-fracture unit (Balzer AG) at −100° C. Replicas of the freshly fractured cells were immediately made by electron beam evaporation of platinum-carbon at angles of 38° and 90° and to thicknesses of 2 and 20 nm. The replicas were incubated overnight in 5% (w/v) SDS to remove cellular material except for those molecules adhering directly to the replicas, washed in distilled water, and incubated briefly in 5% (w/v) bovine serum albumin (BSA) before immunostaining. For immunostaining of freeze-fracture replicas and cryosections, the same primary antibodies as mentioned above were used, followed by donkey anti-guinea pig 18 nm gold conjugate, goat anti-murine 12 nm gold-conjugate or goat anti-rabbit 12 nm gold-conjugate (all from Jackson Immunoresearch), respectively. Additionally, to reveal the cellular distribution of eGFP fusions by means of their eGFP tag, a primary antibody against eGFP raised in rabbit (BD Biosciences) was used. Control specimens, prepared without the first antibody, were essentially free of gold particles.

### 2. Synthesis Examples

### Synthesis Example 1

### Preparation of phaP1-Encoding Constructs

a) Cloning of phaP1 Downstream of the Ace Promoter of pJAM2.

Standard molecular biology protocols were used (Sambrook et al., [21]). All polymerase chain reaction (PCR) products were first cloned into a TA vector (pGEM-T Easy; Promega). Ligation products were first controlled by DNA sequencing and then released by digestion with appropriate restriction enzymes before they were cloned into the expression vector pJAM2 which represents an E. coli-Mycobacterium/Rhodococcus shuttle vector containing the 1.5 kbp ace promoter region (SEQ ID NO:17) (Triccas et al., [29]). For subcloning, restriction enzyme recognition sites (underlined, see below) were incorporated in the sequences of the oligonucleotides. The coding region of PhaP1 (SEQ ID NO:18) was amplified without its native start- and stop codon (582 bp) by PCR from R. eutropha H16 genomic DNA using the oligonucleotides

(SEQ ID NO: 1)

phaP1-5′ (5′-AAAGGATCCATCCTCACCCCGGAACAAGTT-3′)
and

(SEQ ID NO: 2)

phaP1-3′ (5′-AAAGGATCCCGATATGCTTTGCCAACGGAC-3′).

Subsequently, the PCR product was cloned colinear to the ace promoter into the BamHI site of pJAM2. By this a functional in-frame fusion with the first six codons of the amiE gene was generated yielding pJAM2::phaP1. The phaP1 gene in the constructed fusion lacked its own stop codon but contained a stop codon after the His6-tag linker sequence of pJAM2. Therefore, the amino acids SRHHHHHH occurred at the C terminal region of the protein.

b) Construction of the phaP1-egfp and phaP1-lacZ Fusions Expressing Plasmids

A 720-bp fragment representing the complete eGFP gene from Aequoria victoria (SEQ ID NO:20) was amplified without the start codon from plasmid pEGFP-N3 (BD Bioscience Clontech) using PCR primers

(SEQ ID NO: 3)

egfp-5′ (5′-AAATCTAGAGTGAGCAAGGGCGAGGAGCTG-3′)
and

(SEQ ID NO: 4)

egfp-3′ (5′AAATCTAGATTACTTGTACAGCTCGTCCATG-3′),

harbouring the native stop codon (twice underlined). The PCR product was then cloned colinear to the ace promoter and downstream of phaP1 into the XbaI site of pJAM2::phaP1, yielding pJAM2::phaP1-egfp. To investigate the expression and distribution of unfused eGFP in control experiments, the phaP1 portion of pJAM2::phaP1-egfp was released from the expression plasmid by BamHI restriction and relegation, yielding pJAM2::egfp.

For construction of pJAM2::phaP1-lacZ, the 3075-bp coding region of lacZ was amplified from genomic DNA of E. coli S17-1 without its native start codon using PCR primers

(SEQ ID NO: 5)

IacZ-5′ (5′-AAATCTAGAACCATGATTACGGATTCACTGG-3′)
and

(SEQ ID NO: 6)

IacZ-3′ (5′-AAATCTAGATTATTTTTGACACCAGACCAACTG-3′)

harbouring the native stop codon (twice underlined). The PCR product was then cloned colinear to the ace promoter and downstream of phaP1 into the XbaI site of pJAM2::phaP1.

### Synthesis Example 2

### Preparation of Constructs Encoding Perilipin A, tip47, ADRP, Oleosin or Oleosin HD

a) Cloning of the Enhanced gfp (egfp) Downstream of the Ace Promoter of pJAM2.

Standard molecular biology protocols were used [21]. All polymerase chain reaction (PCR) products were first cloned into a TA vector (pGEM-T Easy; Promega), controlled by DNA sequencing and then released by digestion with appropriate restriction enzymes before cloning into expression vectors (see below). To facilitate subcloning, restriction enzyme recognition sites (underlined, see below) were incorporated in the sequence of the oligonucleotides. A 720-base pair (bp) fragment, containing the complete coding sequence of egfp (SEQ ID NO:20) was amplified without the start codon from plasmid pEGFP-N3 (BD Bioscience Clontech) using PCR primers

(SEQ ID NO: 3)

egfp-5′ (5′-AAATCTAGAGTGAGCAAGGGCGAGGAGCTG-3′)
and

(SEQ ID NO: 4)

egfp-3′, (5′AAATCTAGATTACTTGTACAGCTCGTCCATG-3′)

harbouring the native stop codon (twice underlined). The PCR product was then cloned colinear to the ace promoter into the XbaI site of pJAM2, an E. coli-Mycobacteria/Rhodococcus shuttle vector containing the 1.5-kbp ace promoter region [29] (SEQ ID NO:17), to create a functional in-frame fusion with the first six codons of the amiC gene and yielding pJAM2::egfp.

### b) Construction of Lipid Body Protein-eGFP Fusion Expressing Plasmids.

Coding regions of the respective proteins were amplified without their native start- and stop codons, to facilitate generation of functional fusion constructs. The murine perilipin A coding region (1551 bp) (SEQ ID NO:26) was amplified by PCR from retroviral expression vector pSRα MSVtkneo harbouring murine perilipin A cDNA [8] using oligonucleotides

(SEQ ID NO: 7)

perA-5′ (5′-AAAAGTACTTCAATGAACAAGGGCCCAACC-3′)
and

(SEQ ID NO: 8)

perA-3′ (5′-AAAAGTACTGCTCTTCTTGCGCAGCTGGC-3′).

Human TIP47 cDNA (1302 bp) (SEQ ID NO:30) was amplified from plasmid pQE31 [7] using oligonucleotides

(SEQ ID NO:9)

tip47-5′ (5′-AAAGGATCCTCTGCCGACGGGGCAGAGGC-3′)
and

(SEQ ID NO: 10)

tip47-3′ (5′-AAAGGATCCTTTCTTCTCCTCCGGGGCTT-3′).

Human ADRP cDNA (1311 bp) (SEQ ID NO:34) was amplified from an ADRP cDNA fragment provided by C. Londos (Laboratory of cellular and developmental biology, National Institutes of Health, Bethesda) using oligonucleotides

(SEQ ID NO: 11)

adrp-5′ (5′-AAAAGTACTAGTTTTATGCTCAGATCGCTGG-3′)
and

(SEQ ID NO: 12)

adrp-3′ (5′-AAAAGTACTGCATCCGTTGCAGTTGATCCAC-3′).

Each PCR product comprising the PAT family genes was cloned colinear to the ace promoter upstream of the egfp region into the BamHI or ScaI site of pJAM2::egfp, creating pJAM2::perAₘᵤᵣ-egfp, pJAM2::tip47ₕᵤₘ-egfp and pJAM2::adrpₕᵤₘ-egfp, respectively.

A 567-bp fragment representing the cDNA coding region of the 18 kDa maize oleosin (SEQ ID NO:38) was amplified from plasmid pL2± [19] using oligonucleotides

(SEQ ID NO: 13)

oleo-5′ (5′-AAAGGATCCGCGGACCGCGACCGCAGCGG-3′)
and

(SEQ ID NO: 14)

oleo-3′ (5′-AAAGGATCCCGAGGAAGCCCTGCCGCCG-3′)

and was then cloned into the BamHI site of pJAM2::egfp, creating pJAM2::oleo_{mays}-egfp. Similarly, an eGFP fusion with a truncated maize oleosin, representing only its central hydrophobic domain (amino acids 48-113 of SEQ ID NO:39), was constructed by PCR using oligonucleotides

(SEQ ID NO: 15)

oleoHD-5′(5′-AAAGGATCCGCGCTGACGGTGGCGACGCTG-3′)
and

(SEQ ID NO: 16)

oleoHD-3′ (5′-AAAGGATCCCGCCGTGTTGGCGAGGCACGT-3′).

This plasmid was referred to as pJAM2::o/eoHD-egfp. Next, the egfp portion of each fusion was released by XbaI restriction and relegation from each of the constructed expression plasmids, yielding pJAM2: perAₘᵤᵣ, pJAM2:: tip47ₕᵤₘ, pJAM2::adrpₕᵤₘ, pJAM2: oleo_{mays} and pJAM2::oleoHD, respectively. Since the lipid body protein genes in each of the constructed fusion lack their own stop codon but contain one after the His6-tag linker sequence of pJAM2, the amino acids SRHHHHHH were added to the C terminus of the respective proteins.

### 3. Expression Experiments

A. Experiments with phaP1

### Example A1

### Expression of phaP1 in Recombinant Strains of M. smegmatis mc² 155 and R. opacus PD630 and Distribution of the Translation Product in Subcellular Fractions

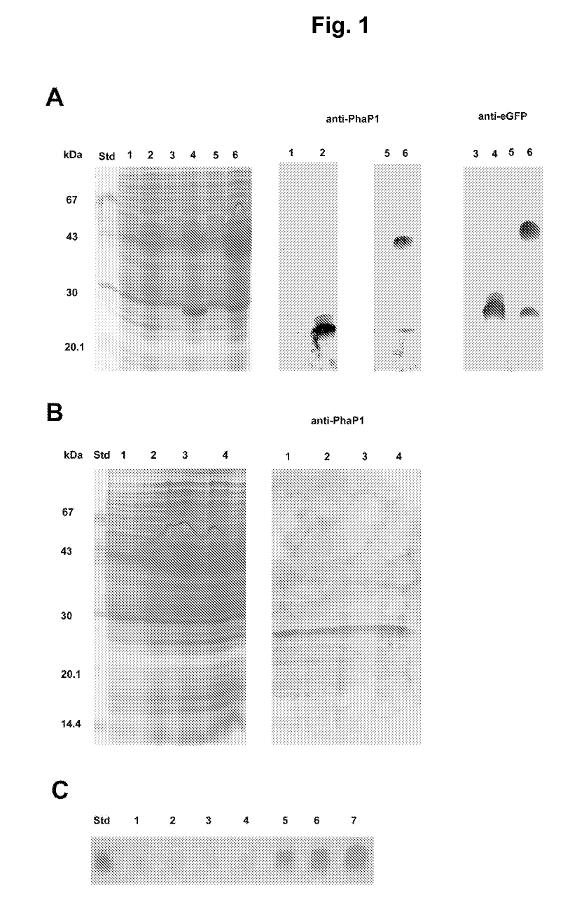

To determine heterologous expression of egfp, phaP1 and the phaP1-egfp in the recombinant actinomycetes, cell crude extracts and cell fractions of cells grown for 72 h under ammonium reduced conditions were analyzed by SDS-PAGE and Western blots as described in the Methods section. Electropherograms of cells of M. smegmatis harbouring pJAM2::phaP1 exhibited an additional protein with an apparent molecular weight of 25 kDa when induced with 0.5% (w/v) acetamide. This molecular weight (M_{W}) corresponded well with that calculated for the His6-tagged PhaP1. The His6-tagged PhaP1 was easily recognized on corresponding Western blots applying anti-PhaP1 IgGs. However, synthesis of His6-tagged PhaP1 was significantly lower compared to the strains synthesizing the 52 kDa PhaP1-eGFP fusion and the unfused 27 kDA eGFP, which was also demonstrated on Western blots using the anti-PhaP1 and anti-eGFP IgGs. All IgGs recognized no proteins in cell crude extracts of the non induced cultures, indicating that in M. smegmatis the synthesis of the recombinant proteins was strictly regulated by the addition of acetamide. As no products of lower M_{W} were detected in the electropherograms and Western blots of cells harbouring pJAM2::phaP1 and pJAM2::egfp, these proteins seemed to be stable against proteolysis in the cytoplasm. However, applying the anti-PhaP1 and anti-eGFP IgGs on crude extracts of M. smegmatis pJAM2::phaP1-egfp revealed that slight cleavage of the fusion protein occurred. In addition, all SDS-PAGE electropherograms of crude extracts of M. smegmatis cells exhibited an additional protein of 44 kDa, which most likely represented the chromosomally encoded acetamidase when cells were induced with 0.5% (w/v) acetamide (FIG. 1 A). The intracellular stability of PhaP1 in recombinant M. smegmatis was also demonstrated by extending the expression time to 96 h (FIG. 1 B).

We tried to determine the distribution of PhaP1 in subcellular fractions of recombinant cells of M. smegmatis. Unfortunately, induction of the cells with acetamide resulted in a severe decrease of TAG accumulation and number of TAG inclusions, even when the concentration of acetamide was reduced to 0.05% (FIG. 1 C). This might be due to cleavage of the inductor by the chromosomally encoded acetamidase, thus providing the cells with sufficient ammonium for growth. Attempts to achieve a sufficient accumulation of TAG inclusions under phosphate limitation in SM as described in the Methods section failed due to the poor growth and little lipid accumulation (data not shown).

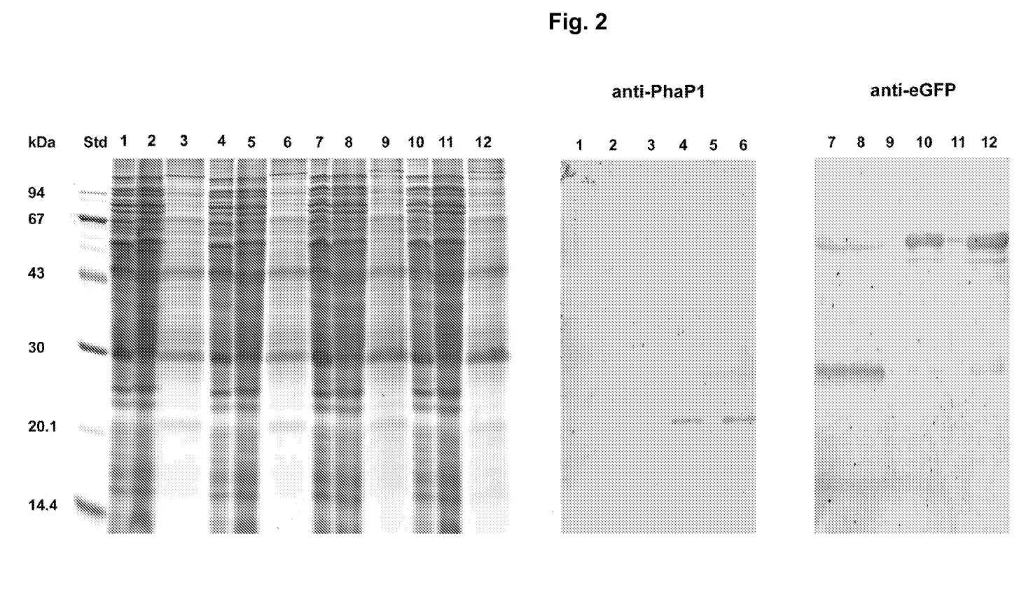

To circumvent this obstacle, all constructed plasmids were subsequently introduced in R. opacus. In contrast to M. smegmatis, SDS-PAGE electropherograms of crude extracts of the corresponding recombinant strains of R. opacus revealed no additional visible protein bands in comparison to crude extracts obtained from the wild type when grown 72 h in ammonium reduced MSM, even when the cells were induced with 0.5% (w/v) acetamide, indicating that expression of genes controlled by the M. smegmatis ace promotor was significantly lower in R. opacus. However, according to the results obtained in recombinant M. smegmatis, the anti-PhaP1 IgGs recognized a 25 kDa protein in Western blots obtained from crude extracts of cells harbouring pJAM2::phaP1, although immunological recognition of the phasin was significantly weaker than in recombinant M. smegmatis. Like in M. smegmatis, no degradation products of the phasin were detected in R. opacus. Similarly, eGFP and the PhaP1-eGFP fusion were easily recognized on Western blots of crude extracts cells harbouring pJAM2::egfp and pJAM2::phaP1-egfp, respectively, as was demonstrated by employing the anti-eGFP IgGs (FIG. 2). In contrast to M. smegmatis, addition of 0.5% (w/v) acetamide to the cultures did not affect TAG accumulation in R. opacus (not shown). To investigate the cellular distribution of PhaP1, eGFP and the PhaP1-eGFP fusion in R. opacus, crude extracts of induced cells were fractionated into soluble fractions, membrane fractions and fractions representing the TAG inclusions. On Western blots of the respective fractions of R. opacus pJAM2::phaP1, the phasin was recognized by the anti-PhaP1 IgGs in the fraction representing the TAG inclusions, whereas no signal occurred in the soluble fraction. This indicated that PhaP1 is associated with the TAG inclusions in recombinant R. opacus. The result obtained for the distribution of PhaP1 in cell fractions of R. opacus was also confirmed by the localization of the PhaP1-eGFP fusion by employing the anti-eGFP IgGs on Western blots of the strain harbouring pJAM2::phaP1-egfp. In this recombinant strain the fusion protein also occurred only in the fraction representing the TAG inclusions. We tried to localize PhaP1 and its eGFP fusion also in electropherograms of total membrane fractions of the recombinant strains, but failed (data not shown). As expected, the unfused eGFP was only localized in the soluble fraction of the control strain harbouring pJAM2::egfp (FIG. 2).

### Example A2

### Distribution of PhaP1-eGFP Fusion Protein in Recombinant R. opacus PD630 and M. smegmatis mc²155

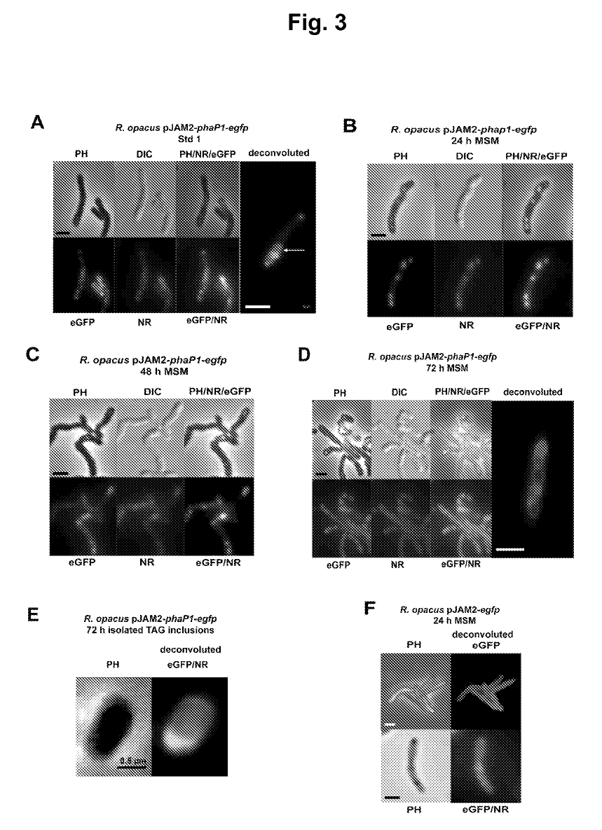

To verify the association of PhaP1-eGFP with the TAG inclusions in R. opacus, the distribution of the fusion protein was investigated by fluorescence microscopy in cells grown in Std1 medium and also for 24, 48 and 72 h in ammonium reduced MSM under conditions permissive for TAG accumulation when formation of large intracellular TAG inclusions occurred in the cytoplasm. The fluorescence of the fusion protein was predominantly associated with TAG inclusions at all stages of their formation. Whereas in cells grown in Std1 medium fluorescence was associated with nascent TAG inclusions at the plasma membrane, it was predominantly associated with matured TAG inclusions in the cytoplasm after growth of the cells in ammonium reduced MSM for 24, 48 and 72 h (FIG. 3 A-D). As revealed by constrained iterative deconvolution of images obtained from Std1 grown cells, fluorescence occurred also to some extent at regions of the cell wall and plasma membrane. However, fluorescence at these sides was much weaker when compared to that of intracellular TAG inclusions (see deconvoluted image in FIG. 3 A). After 72 h in ammonium reduced MSM, cells were fully packed with brightly fluorescent TAG inclusions. Actually, after deconvolution, large TAG inclusions in these cells often exhibited a ring of fluorescence, indicating a localization of the fusion protein at the surface of the inclusions (FIG. 3 D). Fluorescence of the fusion protein was throughoutly distinguishable from Nile Red fluorescence in all stages of TAG accumulation, which, in addition to the TAG inclusions, also clearly labeled the cellular envelope (FIG. 3 A-D).

After disruption of the cells, fluorescence of PhaP1-eGFP was observed in association with isolated TAG inclusions, indicating that the fusion protein was stably associated with the inclusions. Similar to the observation in whole cells, isolated inclusions showed a ring of green fluorescence at their periphery in deconvoluted images (FIG. 3 E). In contrast to this, TAG inclusions from cells expressing unfused eGFP exhibited no fluorescence when observed without Nile Red labeling (not shown). Cells expressing unfused egfp, which served as a negative control, exhibited a diffuse green fluorescence throughout the cytoplasm, whereas intracellular TAG inclusions were easily detectable by their Nile Red fluorescence (FIG. 3 F).

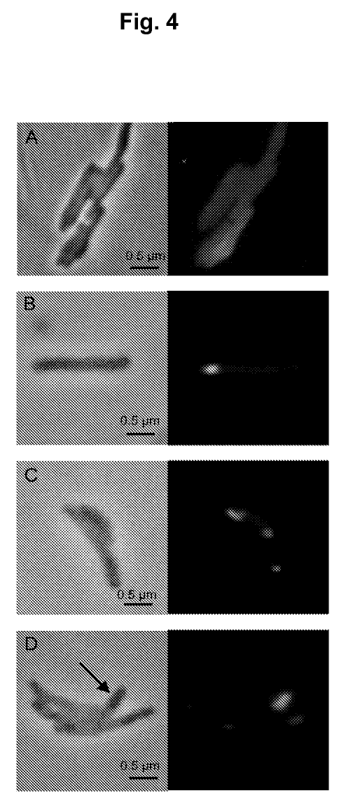

Cells of M. smegmatis mc²155 expressing unfused egfp exhibited a diffuse fluorescence in the cytoplasm similar to that observed in R. opacus PD630 (FIG. 4 A). Corresponding to the results in recombinant R. opacus PD630, in cells of M. smegmatis mc² 155 harbouring pJAM2::phaP1-egfp not induced with acetamide, fluorescence was observed at positions of TAG inclusions at any stage of their formation, indicating that the phasin also targets to the inclusions. However, since the number and size of TAG inclusions in M. smegmatis mc²155 never reached those in R. opacus PD630, TAG inclusions appeared exclusively as discrete points of fluorescence in the cytoplasm (FIG. 4 B-D). In contrast, in cells induced with acetamide a very strong fluorescence appeared in the cells, which could not be related to subcellular structures (data not shown). This is probably due to the abundance of the fusion protein in the cells.

### Example A3

### Immunogold Labeling of Cryosections

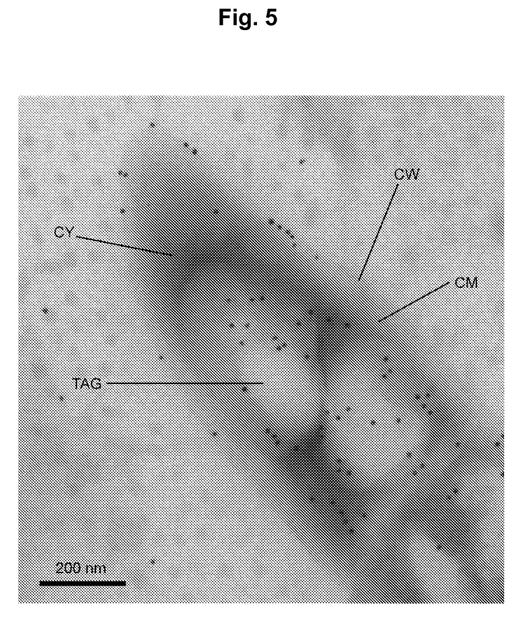

To investigate whether PhaP1-eGFP is targeted exclusively to the surface of TAG inclusions in R. opacus PD630 or also to other components of the cells, the fusion protein was localized on cryosections by postembedding immunogold labeling. Ultrathin cryosections were prepared from recombinant cells grown under storage condition for 72 h. For immunogold labeling of cryosections, rabbit anti-PhaP1 IgGs were used in combination with goat anti-rabbit IgG gold-conjugates. In cryosectioned R. opacus PD630 cells, TAG inclusions appeared as nearly spherical, electron-translucent areas with little internal structure. Strong labels of PhaP1-eGFP were found at the surface of the inclusions, whereas almost no label was observed in the cytoplasm. Label was also detected at the plasma membrane. However, the concentration of PhaP1-eGFP label at the periphery of the cells was lower as compared to that at the surface of the TAG inclusions (FIG. 5).

### Example A4

### Immobilization of E. coli LacZ on TAG Inclusions in R. opacus PD630

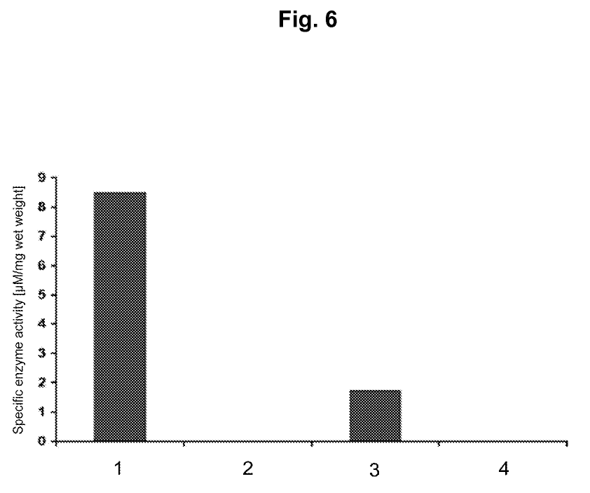

Once the binding of the native PhaP1 and of the PhaP1-eGFP fusion to the TAG inclusions was demonstrated, it was investigated whether PhaP1 could be used as an anchor for immobilization of active enzymes on the surface of TAG inclusions. For this, a fusion of E. coli lacZ as reporter gene to the 3′-terminal region of phaP1 was constructed in plasmid pJAM2. The resulting plasmid pJAM2::phaP1-lacZ was transferred to R. opacus PD630, and the cells were cultivated for 72 h. Subsequently, the TAG inclusions were isolated and used for enzymatic conversion of ONPG. For control experiments, cells harbouring pJAM2::phaP1 were utilized in the same manner. Samples containing TAG inclusions of the control strain exhibited only low β-galactosidase activity. Since R. opacus PD630 expresses also a chromosomally encoded β-galactosidase, low enzyme activity was expected to occur also in the control samples. Furthermore, it was shown that various cytosolic proteins bind unspecifically to the TAG inclusions which are then co-purified with the inclusions (Kalscheuer et al. [10a]; Wältermann & Steinbüchel [31]). However, enzyme activity was significantly higher in samples containing TAG inclusions which were isolated from phaP1-lacZ expressing strains. When TAG inclusions were removed from the assays, conversion of ONPG stopped immediately in all experiments. This result excludes a participation of free β-galactosidase molecules, which were not removed by the purification steps (FIG. 6). These data demonstrate a stable immobilization of LacZ to bacterial TAG inclusions mediated by PhaP1 as an anchor.

### Discussion of Expression Examples

### Part A

In this section of the experimental part it was shown that cells of recombinant strains of R. opacus PD630 and M. smegmatis mc²155 transformed with the R. eutropha H16 phaP1 gene synthesized the phasin PhaP1. The key finding of these experiments is that the phasin remained stable in the cells and that PhaP1 and PhaP1 fusion proteins were targeted to TAG inclusions. This is the first report on the binding of a phasin protein to TAG inclusions. In R. eutropha H16 PhaP1 is strictly associated with the PHB granule fraction, and its expression is highly associated with PHB synthesis due to the regulation exerted by the transcriptional repressor PhaR (Pötter et al. [18d]). The motif in PhaP1, which targets the phasin to PHB granules in R. eutropha H16, has not been identified, yet. However, PHB granules as well as TAG inclusions possess a hydrophobic core of the polyester or lipid, respectively, which is thought to be surrounded by a monolayer of phospholipids (de Koning & Maxwell [6b]; Hocking & Marchessault [9a]; Mayer & Hoppert [16a]; Wältermann et al. [30]). This common structure allows the targeting of PhaP1 to PHB granules and obviously also TAG inclusions as demonstrated in these experiments. Therefore, the present data indicate that targeting of PhaP1 to PHB granules in R. eutropha H16 is most probably not mediated by a direct mutual recognition of the phasin and the polymer in the granules. The results indicate that PhaP1 has obviously the ability to bind to any type of hydrophobic inclusion, irrespectively whether a PHA or a different hydrophobic compound is present in the core of the inclusions. Furthermore, it is also not probable that an additional, not yet identified component involved in PHA metabolism mediates targeting of PhaP1 to the inclusions, since such components should be absent in the strains used in our study. Most probably, binding of PhaP1 to the inclusions is mediated only by the presence of the amphiphilic interphase consisting of the monolayer membrane between the inclusions and the surrounding cytoplasm or by the hydrophobic surface of the core or by a combination of both.

Combined electron microscopy and postembedding immunocytochemistry revealed that PhaP1 is distributed mostly on the amphiphilic surface of the TAG inclusions. However, in contrast to its exclusive distribution on the surface of PHB granules in R. eutropha H16, it was demonstrated that some PhaP1 was also present at the plasma membrane and cell wall regions in cells of R. opacus PD630. This distribution was also reported by Pieper-Fürst et al. [18a]) while investigating the cellular distribution of the 14 kDa phasin in Rhodococcus ruber, which is able to synthesize equal amounts of TAGs and of the copolymer poly(3-hydroxybutyrate-co-3-hydroxyvalerate). Although it is unknown whether in R. ruber TAGs and poly(3HB-co-3HV) occur separately or simultaneously in the inclusions, it was demonstrated that in this strain the phasin occurs on the surface of any inclusion in the cells and also at the cytoplasmic site of the plasma membrane. According to a recently proposed model, the origin of TAG inclusions in prokaryotes is the cytoplasmic site of the plasma membrane (Wältermann et al. [31]). Thus, a binding of phasins to nascent TAG inclusions at their site of synthesis is the most probable explanation for this distribution.

In R. eutropha H16, the amount of PHB and the number of granules is directly influenced by the amount of phasin molecules in the cells (Wieczorek et al. [31a]; Pötter et al. [18d]). The presence of the phasin did neither alter the amount of TAGs in R. opacus PD630 or influence the size or number of TAG inclusions. As revealed by the present expression analysis, the total amount of PhaP1 in the cells was very low, since expression of the protein was limited by the ace promoter of plasmid pJAM2. Whether the presence of a high amount of phasins could influence TAG metabolism in the cells remains to be elucidated.

It was demonstrated that TAG inclusions tagged with a PhaP1-LacZ fusion exhibited β-galactosidase activity in vitro. Immobilization of enzymes and other kind of proteins on surfaces or defined particles offers interesting applications. One example could be the synthesis of functionalized nanoparticles, for example such carrying antibodies for analytic purposes or hormones and other therapeutics. Such nanoparticles can be purified easily from cell crude extracts. Moldes et al. [17a] created a system for the synthesis and purification of enzymes using PHA granules as matrix and the N-terminus of the phasin PhaF from Pseudomonas putida as linker. Furthermore, PHB granules in recombinant E. coli have been successfully demonstrated as matrix for the purification of target proteins by fusions with phasins and self-cleaving affinity tags based on protein splicing elements known as inteins (Banki et al. [3a]). Also TAG inclusions were utilized as matrix for purification of enzymes by Moloney [17c; 17d]. The author created a system based on plant cells by attaching target enzymes to oil bodies via oleosins. Both purification systems described above are patented and commercially available (Prieto et al.: ES patent 200102240 [18f]; Moloney: U.S. Pat. Nos. 5,650,554 [17b] and 6,924,363 [17e]). In addition, anchoring of enzymes and other proteins to TAG inclusions by a PhaP1 tag offers an interesting possibility to establish alternative, bioengineered pathways on the monolayer surface of intracellular TAG inclusions.

B. Experiments with Eukaryotic Lipid Body Protein

### Example B1

### Expression of Eukaryotic Lipid Body Proteins in Recombinant Actinomycetes

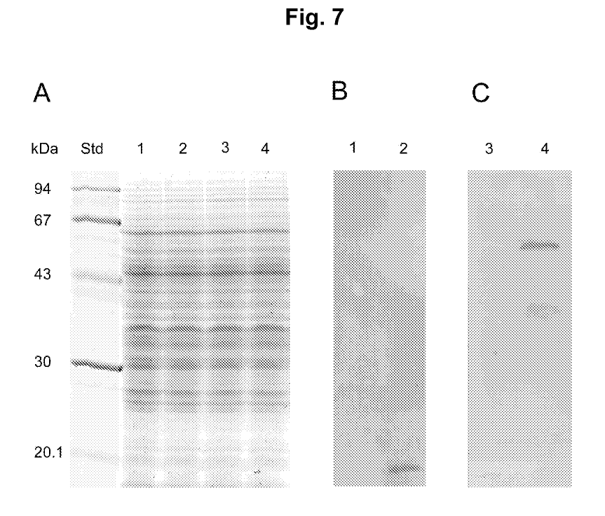

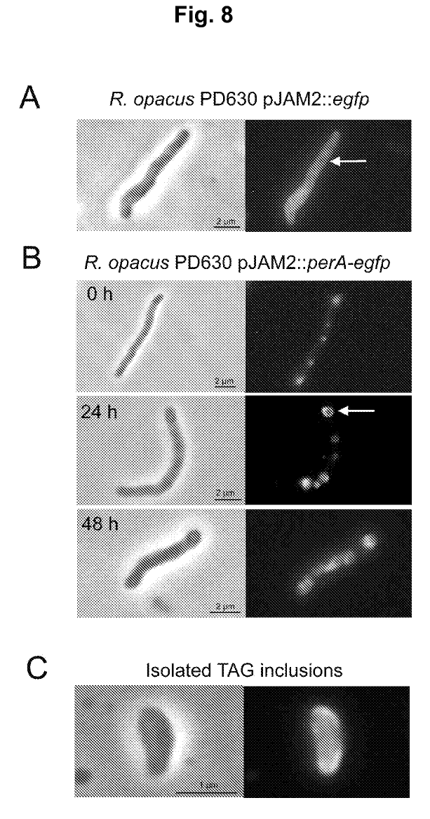

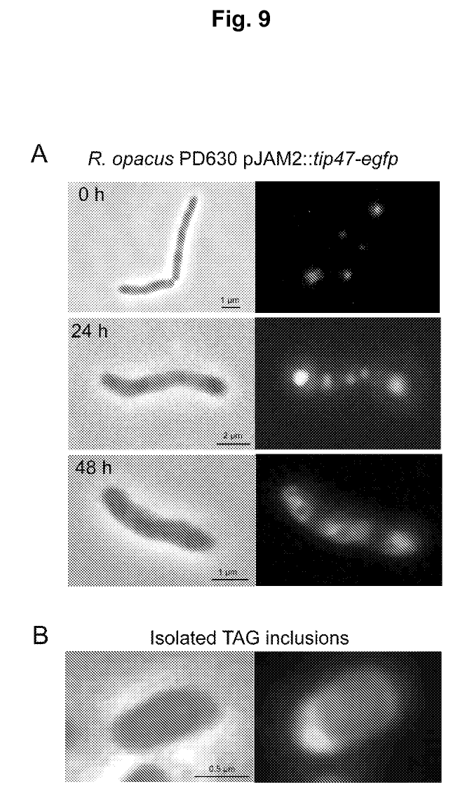

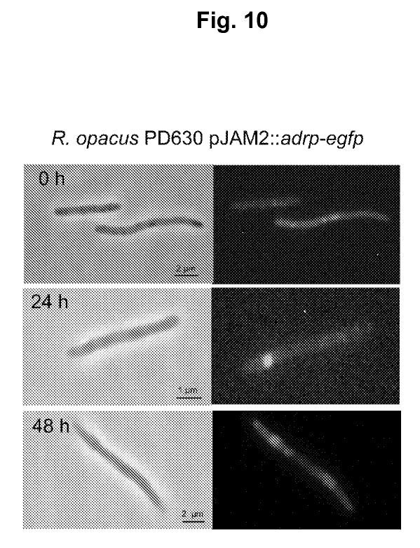

The coding regions of murine perilipin A (SEQ ID NO:26), human ADRP (SEQ ID NO:34), human TIP47 and maize oleosin (SEQ ID NO:38) genes were cloned as His6-tagged fusions into the E. coli-Rhodococcus/Mycobacterium shuttle vector pJAM2. Crude extracts of the respective transformed M. smegmatis mc²155 and R. opacus PD630 cells were analyzed for their perilipin A, ADRP, TIP47 and oleosin expression by SDS-PAGE and immunoblotting, using the antibodies listed in the Materials and Methods section. All antibodies did not recognize any protein in untransformed Rhodococcus/Mycobacterium cells. The chicken anti-maize oleosin IgG easily recognized a 19-kDa protein in M. smegmatis mc²155 cells transformed with pJAM2::oleo_{mays} (FIG. 7). However, in cells of R. opacus PD630 harbouring pJAM2::oleo_{mays} expression was significantly lower and only observable on overexposed immunoblots, even if compared to M. smegmatis mc²155 cells not induced with acetamide (not shown). This 19-kDa protein should be the His6-tagged oleosin derived from the maize gene in pJAM2::oleo_{maize}. No proteolytic degradation products of lower Mᵣ were detected in R. opacus PD630 and M. smegmatis mc²155, indicating that the protein was stable against intracellular proteolysis. Expression of the His6-tagged murine perilipin A in M. smegmatis mc²155 and R. opacus PD630 harbouring plasmid pJAM2::perAₘᵤᵣ resulted in a single signal of 58 kDa on immunoblots, with a similar intensity to that of recombinant oleosin expression, indicating that the protein was also stable and that intracellular proteolysis did not occur (FIG. 7). In crude extracts of M. smegmatis mc²155 and R. opacus PD630 harbouring pJAM2::tip47ₕᵤₘ or pJAM2::adrpₕᵤₘ, respectively, no observable synthesized protein could be detected on immunoblots. Thin layer chromatography and fluorescence microscopy using Nile Red as a dye revealed that presence of the plasmids and expression of the proteins did not alter the lipid content of the cells or the number, shape or size of the lipid inclusions as compared to the wild types in absence of acetamide. In contrast to this, cells of M. smegmatis mc²155 contained a significant decreased amount of TAGs and number of lipid inclusions, when more than 0.01% (w/v) acetamide was added to the cultures (not shown).

### Example B2

### Fluorescence Localization of PAT Protein- and Oleosin Fusions in Recombinant R. opacus PD630 and M. smegmatis mc²155

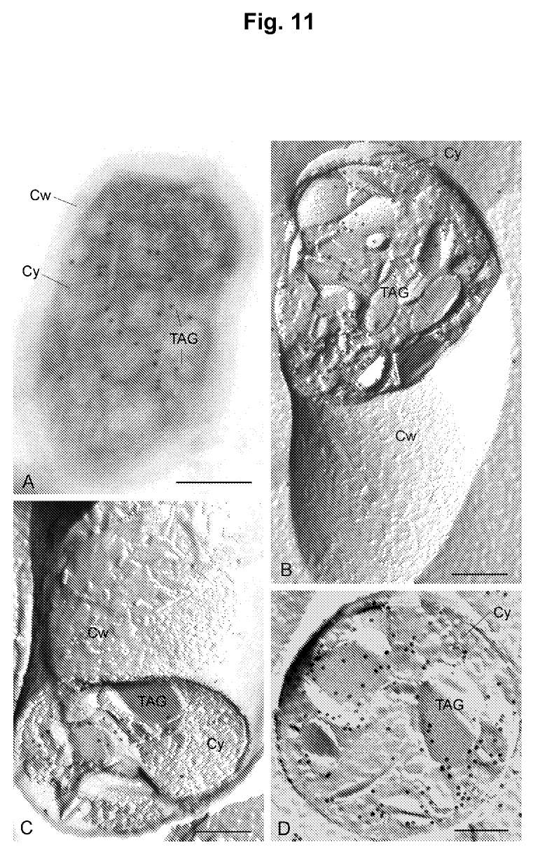

Experiments were performed to localize perilipin A and maize oleosin in subcellular fractions of recombinant R. opacus PD630 by immunoblot analysis, but failed due to the small amounts of protein that were synthesized and the low sensitivity of the immunoblot assay. To reveal the subcellular localization and binding properties of PAT proteins and the oleosin to bacterial TAG inclusions, the lipid body proteins were visualized in recombinant strains as fusions with eGFP. Cells of R. opacus PD630 and M. smegmatis mc²155 transformed with the respective PAT protein-eGFP fusion expression plasmids were cultivated for 0, 24 and 48 h under storage conditions and inspected for their fluorescence pattern. Cells harbouring plasmid pJAM2::egfp expressing unfused eGFP served as a negative control. Under these conditions and during these periods of time, cells increased their lipid content and accumulated large amounts of lipid inclusions in the cytoplasm, which were derived from peripheral lipid domains according to earlier observations [6, 30]. Unfused eGFP showed a broad and diffuse fluorescence throughout the cytoplasm in R. opacus PD630 and M. smegmatis mc²155. However, images obtained from M. smegmatis mc²155 were poor compared to those obtained from R. opacus PD630 due to its distinct confluent growth, but corresponded well to all the results obtained in recombinant strains of R. opacus PD630. The fluorescence was excluded from large lipid inclusions occurring in later stages of lipid accumulation (FIG. 8 A). In contrast, strains harbouring pJAM2::perAₘᵤᵣ-egfp exhibited fluorescence exclusively in small lipid inclusions attached to the plasma membrane in early stage of lipid accumulation. During proceeding TAG accumulation and formation of cytoplasmic lipid inclusions, perilipin A-eGFP fluorescence appeared to be associated with cytoplasmic lipid inclusions often observed as peripheral rings surrounding the inclusions (FIG. 8 B). To reveal if this fluorescence pattern was not resulting from of simple exclusion of perilipin A-eGFP fluorescence from the lipid inclusions, the lipid inclusions were isolated from the respective recombinant R. opacus PD630 strains and investigated in fluorescence microscopy. In addition, the lipids in the core of the inclusions were stained with Nile Red. Isolated inclusions from perilipin A-eGFP expressing cells exhibited a clear ring shaped fluorescence at their surface, with red fluorescence of the lipid core caused by the incorporated Nile Red dye (FIG. 8 C). In contrast, lipid inclusions in cells expressing unfused eGFP exhibited no fluorescence when observed without Nile Red labeling. These data indicate that perilipin A-eGFP associates closely with the surface of lipid inclusions in recombinant R. opacus PD630 and remains also stably associated during the cell disruption process.

Time-laps experiments testing the subcellular localization of ADRP-eGFP and TIP47-eGFP in recombinant R. opacus PD630 strains harbouring pJAM2::adrpₕᵤₘ-egfp or pJAM2::tip47ₕᵤₘ-egfp, respectively, were also performed. Both recombinant strains synthesized lipid inclusions similar to those observed in the wild type and the perilipin A-eGFP expressing strain. In contrast to the immunoblot analysis, clear fluorescence was observable in R. opacus PD630 and M. smegmatis mc²155 harbouring pJAM2::tip47ₕᵤₘ-egfp. The fluorescence was exclusively localized to intracellular, peripheral lipid domains at the beginning of lipid accumulation. After 24 and 48 h under storage conditions, large cytoplasmic lipid inclusions occurred. Similarly to the results obtained in R. opacus PD630 synthesizing perilipin A-eGFP, TIP47-eGFP fluorescence was often observed in the form of rings surrounding large lipid inclusions (FIG. 9 A). This labeling pattern was also confirmed on isolated inclusions tagged with TIP47-eGFP (FIG. 9 B). In strains harbouring pJAM2::adrpₕᵤₘ-egfp fluorescence was very weak in early stages of lipid accumulation, but clearly distinguishable from auto fluorescence in control experiments performed with wild type R. opacus PD630. ADRP-eGFP was clearly visible in lipid inclusions after 24 and 48 h of lipid accumulation. However, background fluorescence was also observed, which might be due to prolonged exposure time during image recording (FIG. 10).

### Example B3

### Immunogold Labeling of Cryosections and Freeze-Fracture Replicas of Recombinant R. opacus PD630

To verify the exclusive localization of PAT family proteins on intracellular TAG inclusions in R. opacus PD630 and M. smegmatis mc²155 as revealed by the fluorescence microscopic investigations, postembedding immunogold labeling on cryosections was performed using antibodies raised against the PAT family proteins and the eGFPtag listed in the Materials and Methods section. However, immunogold labeled cryosections of recombinant cells of R. opacus PD630 and M. smegmatis mc²155 expressing eGFP fusions of perilipin A or ADRP were indistinguishable from the respective control experiments, which, in case of ADRP, might be due to the low amount of protein synthesized. Only experiments using the guinea pig anti-human TIP47 antibodies yielded reliable and fine results, and corresponding to our preceeding observations, TIP47-eGFP was exclusively associated with the TAG inclusions in R. opacus PD630 harbouring pJAM2::tip47ₕᵤₘᵣ-egfp (FIG. 11 A).

Since formation of TAG inclusions in bacteria is an emulsion aggregation driven process, which could cause an encapsulation of lipid-binding proteins into the lipid core, freeze-fracture experiments were carried out to reveal the distribution of PAT family proteins on the surface and core of TAG inclusions in the recombinant cells. In general, when bacterial cells are freeze-fractured, the fracture plane runs between both leaflets of cellular membranes. Sometimes, the fracture plane runs across the cells and intracellular lipid inclusions, enabling a cross-fractured view into the core of the inclusions. In freeze-fracture replicas of R. opacus PD630, a series of tightly compressed, alternately oriented lipid layers of varying depths, appeared throughout the fractured core of TAG inclusions, similar to that previously observed in cross-fractured eukaryotic and prokaryotic TAG inclusions [20, 30]. The outermost of these layers is thought to originate from the surrounding phospholipid layer. For immunogold labeling of PAT proteins in freeze-fracture replicas, recombinant cells of R. opacus PD630 were grown under storage conditions for 48 h. Corresponding to the labeling experiments on cryosections, labeled replicas of ADRP and perilipin A expressing cells of R. opacus PD630 showed no reliable results and were indistinguishable from the respective controls. However, after labeling of the replicas obtained from the strain harbouring pJAM2::tip47ₕᵤₘ, a variety of locations within the lipid inclusions were labeled (FIG. 11B+C). No significant labeling of the surroundings of the cells, the cytoplasm and the different faces of the plasma membrane were obtained. The distribution of TIP47 in recombinant R. opacus PD630 was also confirmed on replicas of the respective strain expressing the eGFP-tagged TIP47, in which labeling was performed using rabbit anti-eGFP IgGs as the primary antibody (FIG. 11 C).

### Discussion of Expression Examples

### Part B

In this section of the experiments it was demonstrated the synthesis of the mammalian lipid body proteins perilipin A, ADRP and TIP47 in TAG accumulating actinomycetes and their targeting to intracellular TAG inclusions. Perilipins and ADRP were previously exclusively found associated with lipid bodies in eukaryotic cells, but the mechanisms by which they are targeted to the lipid bodies remained unclear [5]. One of the most intriguing results of the localization experiments in this study is that the coating of preexisting lipid bodies with PAT proteins occurred in vivo via the cytoplasm. Furthermore, PAT family proteins must interact directly with the lipids, because an indirect anchorage mediated by other specific proteins can be excluded because they were absent in the prokaryotic systems. Sequences for targeting of a few lipid droplet proteins have been reported in the literature. For example, the targeting and anchorage of perilipins are assumed to be mediated by three hydrophobic sequences in the central 25% region of the protein, although the exact targeting mechanism remains to be elucidated [25]. Freeze-fracture immunogold labeling showed that TIP47 was not only present on the amphipathic surface but also in the hydrophobic core of the TAG inclusions in recombinant R. opacus PD630. This distribution pattern must be due to the special mechanism by which lipid inclusions in bacteria are formed. In bacteria, TAGs are synthesized as small WS/DGAT-associated droplets forming an oleogenous, emulsive layer at the plasma membrane that aggregate/coalesce to lipid prebodies and are then released to form cytoplasmically localized lipid inclusions during proceeding lipid synthesis [30]. By association of the PAT proteins with uncoated lipids, an encapsulation of PAT proteins could occur during the aggregation/coalescence process of lipids resulting in a capturing of PAT proteins in the hydrophobic core of the inclusions. Therefore, the present findings confirm the current model for the formation of neutral lipid inclusions in bacteria.

The present experiments demonstrate that studies on the formation of bacterial lipid inclusions and targeting of eukaryotic lipid body proteins to these lipid inclusions are a suitable tool to reveal their underlying mechanisms. In addition, targeting molecules like the PAT family proteins could be used as linkers to anchor biotechnologically relevant enzymes on the surface of bacterial lipid inclusions, which could be tailored for a variety of biotechnological applications.

The subsequent table lists all amino acid and nucleic acid sequences referred to in the present description and claims

### LIST OF SEQUENCES

| | NA | AA |
|---|---|---|
| ace | 17 | — |
| ADRP | 34 | 35 |
| ADRP-eGFP | 36 | 37 |
| eGEP | 20 | 21 |
| Oleosin | 38 | 39 |
| Oleosin-eGFP | 40 | 41 |
| Perilipin A | 26 | 27 |
| Perilipin A-eGFP | 28 | 29 |
| phaP1 | 18 | 19 |
| phaP1-eGFP | 22 | 23 |
| phaP1-LacZ | 24 | 25 |
| TIP47 | 30 | 31 |
| TIP47-eGFP | 32 | 33 |
| ispA | 42 | 43 |
| crtE | 46 | 47 |
| crtB | 44 | 45 |
| crtl | 48 | 49 |

AA: Amino Acid Sequence No.
NA: Nucleic Acid Sequence No.

The present invention is not limited to the above-mentioned specific sequences. It is understood that the present invention also encompasses additional sequences derived from the above.

A “derived” sequence, e.g. a derived amino acid or nucleic acid sequence, means, according to the invention, unless stated otherwise, a sequence that has identity of at least 80% or at least 90%, in particular 91%, 92%, 93%, 94%, 95%, 96%, 97%, 98% and 99%, with the starting sequence.

### REFERENCES

1. Abell, B. M., L. A. Holbrook, M. Abenes, D. J. Murphy, M. J. Hills, and M. M. Moloney. 1997. Role of the proline knot motif in oleosin endoplasmic reticulum topology and oil body targeting. Plant Cell 9:1481-1493
2. Alvarez, H. M., F. Mayer, D. Fabritius, and A. Steinbüchel. 1996. Formation of intracytoplasmic lipid inclusions by Rhodococcus opacus PD630. Arch. Microbiol. 165:377-386
3. Alvarez, H. M., and A. Steinbüchel. 2002. Triacylglycerols in prokaryotic microorganisms. Appl. Microbiol. Biotechnol. 60:367-376
3a. Banki, M. R., Gerngross, T. U. & Wood D. W. (2005). Novel and economical purification of recombinant proteins: Intein-mediated protein purification using in vivo polyhydroxybutyrate (PHB) matrix association. Prot. Sci. 14, 1387-1395.
4. Barbero, P., E. Buell, S. Zulley, and S. R. Pfeffer. 2001. TIP47 is not a component of lipid droplets. J. Biol. Chem. 276:24348-24351
5. Brown, D. A. 2001. Lipid droplets. proteins floating on a pool of fat. Curr. Biol. 11:446-449
6. Christensen, H., N. J. Garton, R. W. Horobin, D. E. Minnikin, and M. R. Barer. 1999. Lipid domains of mycobacteria studied with fluorescent molecular probes. Mol. Microbiol. 31:1561-1572
6a. Darzins, E. (1958). The bacteriology of tuberculosis. Minneapolis, Minn.: University of Minnesota Press.
6b. de Koning, G. J. M. & Maxwell I. A. (1993). Biosynthesis of poly-(R)-3-hydroxyalkanoate: an emulsion polymerization. J. Environ. Degrad. 1, 223-226.
7. Diaz, E., and S. R. Pfeffer. 1998. TIP47: a cargo selection device for mannose 6-phosphate receptor trafficking. Cell 93:433-443
8. Garcia, A., A. Sekowski, V. Subramanian, and D. L. Brasaemle. 2003. The central domain is required to target and anchor perilipin A to lipid droplets. J. Biol. Chem. 278:625-635
8a. Gerngross, T. U., Reilly, P., Stubbe, J., Sinskey, A. J. & Peoples, O. P. (1993). Immunocytochemical analysis of poly-β-hydroxybutyrate (PHB) synthase in Alcaligenes eutrophus H16: Localization of the synthase enzyme at the surface of PHB granules. J. Bacteriol. 175, 5289-5293.
9. Greenberg, A. S., J. J. Egan, S. Wek, M. C. Moos, C. Londos, and A. R. Kimmel. 1993. Isolation of cDNAs of perilipin A and perilipin B—sequence and expression of lipid-droplet associated proteins of adipocytes. Proc. Nat. Acad. Sci. USA 90:12035-12039
9a. Hocking, P. J. & Marchessault, R. H. (1994). Biopolyesters. In Chemistry and technology for biodegradable polymers, pp. 48-96. Edited by G. Griffin. London: Chapman and Hall.
10. Kalscheuer, R., M. Arenskötter, and A. Steinbüchel. 1999. Establishment of a gene transfer system for Rhodococcus opacus PD630 based on electroporation and its application for recombinant biosynthesis of poly(3-hydroxyalkanoic acids). Appl. Microbiol. Biotechnol. 52:508-515
10a. Kalscheuer, R., Wältermann, M., Alvarez, H. M. & Steinbüchel A. (2001). Preparative isolation of lipid inclusions from Rhodococcus opacus PD630 and Rhodococcus ruber and identification of granule-associated proteins. Arch. Microbiol. 177, 20-28.
11. Kalscheuer, R., and A. Steinbüchel. 2003. A novel bifunctional wax ester synthase/acyl-CoA:diacylglycerol acyltransferase mediates wax ester and triacylglycerol biosynthesis in Acinetobacter calcoaceticus ADP1. J. Biol. Chem. 287:8075-8082
12. Kalscheuer, R., T. Stöveken, H. Luftmann, U. Malkus, R. Reichelt, and A. Steinbüchel. 2005. Neutral lipid biosynthesis in engineered Escherichia coli: Jojoba like wax esters and fatty acid butyl esters. Appl. Environ. Microbiol. 72:1373-1379
13. Lacey, D. J., J. Wellner, F. Beaudoin, J. A. Napier, and P. R. Shewry. 1998. Secondary structure of oleosins in oil bodies isolated from seeds of safflower (Carthamus tinctorius L.) and sunflower (Helianthus annuus L.). Biochem. J. 334:469-477
14. Lee, W. S., J. T. C. Tzen, J. C. Kridl, S. E. Radke, and A. H. C. Huang. 1991. Maize oleosin is correctly targeted to seed oil bodies in Brassica napus transformed with the maize oleosin gene. Proc. Natl. Acad. Sci. U.S.A. 88:6181-6185
15. Londos, C., D. L. Brasaemle, C. J. Schultz, J. P. Segrest, and A. R. Kimmel. 1999. Perilipins, ADRP, and other proteins that associate with intracellular neutral lipid droplets in animal cells. Semin. Cell Dev. Biol. 10:51-58
16. Lu, X., J. Grucia-Gray, N. G. Copeland, D. J. Gilbert, N. A. Jenkins, C. Londos, and A. R. Kimmel. 2001. The murine perilipin gene: the lipid-droplet-associated perilipins derive from tissue-specific, mRNA splice variants and define a gene family of ancient origin. Mamm. Genome 12:741-749
16a. Mayer, F. & Hoppert, M. (1997). Determination of the thickness of the boundary layer surrounding bacterial PHA inclusion bodies, and implication for models describing the molecular architecture of this layer. J. Basic Microbiol. 37, 45-52.
17. Miura, S., J. W. Gan, J. Brzostowski, M. J. Parisi, C. J. Schultz, C. Londos, B. Oliver, and A. R. Kimmel. 2002. Functional conservation for lipid storage droplet association among perilipin, ADRP, and TIP47 (PAT)-related proteins in mammals, Drosophila and Dictyostelium. J. Biol. Chem. 277:32253-32257
17a. Moldes, C., Garcia, P., Garcia, J. L. & Prieto, M. A. (2004). In vivo immobilization of fusion proteins on bioplastics by the novel tag bioF. Appl. Environ. Microbiol. 70, 3205-3212.
17b. Moloney, M. M. (1997). Oil body proteins as carriers of high-value peptides in plants. U.S. Pat. No. 5,650,554.
17c. Moloney, M. M. (1998). Oleosins as carrier for foreign protein in plant seeds. In Engineering crops for industrial end uses, pp. 47-54. Edited by P. R. Shewry, J. A. Napier & P. Davis. London: Portland Press.
17d. Moloney, M. M. (2002). Oleosin partitioning technology for production of recombinant proteins in oil seeds. In Handbook of industrial culture: mammalian, microbial, and plant cells, pp. 279-298. Edited by V. A. Vinci & S. R. Parekh. Totowa: Humana Press.
17e. Moloney, M. M., Boothe, J. & van Rooijen, G. J. (2005). Oil bodies and associated proteins as affinity matrices. U.S. Pat. No. 6,924,363.
18. Murphy, D. J. 2001. The biogenesis and function of lipid bodies in animals, plants and microorganisms. Prog. Lipid Res. 40:325-438
18a. Pieper-Fürst, U., Madkour, M. H., Mayer, F. & Steinbüchel, A. (1994). Purification and characterization of a 14-kilodalton protein that is bound to the surface of polyhydroxyalkanoic acid granules in Rhodococcus ruber. J. Bacteriol. 176, 4328-4337.
18b. Pieper-Fürst, U., Madkour, M. H., Mayer, F. & Steinbüchel, A. (1995). Identification of the region of a 14-kilodalton protein of Rhodococcus ruber that is responsible for the binding of this phasin to polyhydroxyalkanoic acid granules. J. Bacteriol. 177, 2513-2523.
18c. Pötter, M. & Steinbüchel, A (2005). Poly(3-hydroxybutyrate) granule-associated proteins: Impacts on poly(3-hydroxybutyrate) synthesis and degradation. Biomacromolecules 6, 552-560.
18d. Pötter, M., Madkur, M. H., Mayer, F. & Steinbüchel, A. (2002). Regulation of phasin expression and polyhydroxyalkanoate PHA granule formation in Ralstonia eutropha H16. Microbiology 148, 2413-2426.
18e. Pötter, M., Müller, H., Reinecke, F., Wieczorek, R., Fricke, F., Bowien, B., Friedrich, B. & Steinbüchel, A. (2004). The complex structure of polyhydroxybutyrate (PHB) granules: four orthologous and paralogous phasins occur in Ralstonia eutropha. Microbiology 150, 2301-1311.
18f. Prieto, M. A., Moldes, T. C., Garcia, G. P. & Garcia, L. J. L. (2004). Proteinas de fusion imvilizadas en granulos de polyhydroxyalkanoato de cadena media. ES Patent 200102240.
19. Qu, R. D., and A. H. C. Huang. 1990. Oleosin KD18 on the surface of oil bodies in maize. Genomic and cDNA sequences and the deduced protein structure. J. Biol. Chem. 265:2238-2243
20. Robenek, H., M. J. Robenek, and D. Troyer. 2005. PAT family proteins pervade lipid droplet cores. J. Lipid Res. 46:1331-1338
21. Sambrook, J., E. F. Fritsch, and T. Maniatis. 1989. Molecular cloning: a laboratory manual, p.A. 1, 2^{nd} ed. Cold Spring Harbour Laboratory, Cold Spring Harbour, New York
22. Schlegel, H. G., H. Kaltwasser, and G. Gottschalk. 1961. Ein Submersverfahren zur Kultur wasserstoffoxidierender Bakterien: Wachstumsphysiologische Untersuchungen. Arch. Mikrobiol. 38:209-222
22a. Simon, R., Priefer, U. & Pühler, A. (1983). A broad host range mobilization system for in vivo genetic engineering: transposon mutagenesis in Gram negative bacteria. Biotechnology 1, 784-791.
23. Snapper, S. B., R. E. Melton, S. Mustafa, T. Kieser, and W. R. Jacobs. 1990. Isolation and characterization of efficient plasmid transformation mutants of Mycobacterium smegmatis. Mol. Microbiol. 4:1911-1919
24. Steinbüchel, A. (1991). Polyhydroxyalkanoic acids. In Biomaterials, pp. 123-213. Edited by D. Byrom. London: Macmillan.
24a. Steinbüchel, A. 1996. PHB and other polyhydroxyalkanoic acids, p. 403-464. In H. J. Rehm, G. Reed, A. Pühler, and P. Stadler (ed.), Biotechnology 2^{nd} ed, vol. 6, Wiley VCH, Heidelberg
24b. Steinbüchel, A., Aertz, A., Babel, W., F öllner, C., Liebergesell. M., Madkour, M. H., Mayer, F., Pieper-Fürst, U., Pries, A., Valentin, H. E. & Wieczorek, R. (1995). Considerations on the structure and biochemistry of bacterial polyhydroxyalkanoic acid inclusions. Can. J. Microbiol. 41 (Suppl. 1), 94-105.
24c. Stubbe, J. & Tian, J. (2003). Polyhydroxyalkanoate (PHA) homeostasis: the role of the PHA synthase. Nat. Prod. Rep. 20, 445-457.
25. Subramanian, V., A. Garcia, A. Sekowski, and D. L. Brasaemle. 2004. Hydrophobic sequences target and anchor perilipin A to lipid droplets. J. Lipid Res. 45:1983-1991
26. Ting, J. T. L., R. A. Balsamo, C. Ratnayake, and A. H. C. Huang. 1997. Oleosin of plant seed oil body is correctly targeted to the lipid bodies in transformed yeast. J. Biol. Chem. 272:3699-3705
27. Tokuyasu, K. T. 1980. Immunocytochemistry on ultrathin frozen sections. Histochem. J. 12:381-403
28. Towbin, H., T. Staehelin, and J. Gordon. 1979. Electrophoretic transfer of proteins from polyacrylamide gels to nitrocellulose sheets: procedure and some applications. Proc. Natl. Acad. Sci. USA 76:4350-4354
29. Triccas, J. A., T. Parish, W. J. Britton, and B. Giquel. 1998. An inducible expression system permitting the efficient purification of a recombinant antigen from Mycobacterium smegmatis. FEMS Microbiol. Lett. 167:151-156
30. Wältermann, M., A. Hinz, H. Robenek, D. Troyer, R. Reichelt, U. Malkus, H. J. Galla, R. Kalscheuer, T. Stöveken, P. von Landenberg, and A. Steinbüchel. 2005. Mechanism of lipid-body formation in prokaryotes: how bacteria fatten up. Mol. Microbiol. 55:750-763
31. Wältermann, M., and A. Steinbüchel. 2005. Neutral lipid bodies in prokaryotes: Recent insights into structure, formation, and relationship to eukaryotic lipid depots. J. Bacteriol. 187:3607-3616
31a. Wieczorek, R., Pries, A., Steinbüchel, A. & Mayer, F. (1995). Analysis of a 24-kilodalton protein associated with the polyhydroxyalkanoic acid granules in Alcaligenes eutrophus. J. Bacteriol. 177, 2425-2435.
32. York, G. M., Stubbe, J. & Sinskey, A. J. (2002). The Ralstonia eutropha PhaR protein couples synthesis of the PhaP phasin to the presence of polyhydroxybutyrate in cells and promotes polyhydroxybutyrate production. J. Bacteriol. 184, 59-66.

1. A method of targeting a protein of interest to an intracellular hydrophobic inclusion body of a bacterial cell, comprising expressing a nucleotide sequence encoding a fusion protein comprising a hydrophobic targeting peptide operatively linked with a protein of interest in a bacterial cell carrying a hydrophobic inclusion body.

2. The method of claim 1, wherein said inclusion body is of the TAG-, WE- or PHA-type.

3. The method of claim 1, wherein the targeting peptide is a pro- or eukaryotic peptide.

4. The method of claim 1, wherein the targeting molecule is selected from peptides of bacterial, animal or plant origin.

5. The method of claim 1, wherein the targeting molecule is
a) derived from a protein associated in its native state with prokaryotic PHA inclusion bodies; or
b) derived from a protein associated in its native state with eukaryotic TAG or WE inclusion bodies.

6. The method of claim 5, wherein the targeting molecule is selected from poly hydroxyalkanoate body binding phasins, perilipins, Adipose Differentiation Related Proteins (ADRPs)/adipophilins and Tail Interacting Proteins (TIPs).

7. The method of claim 6, wherein the targeting molecule is selected from:
a) PhaP1 (SEQ ID NO:19),
b) Perilipin A (SEQ ID NO:27),
c) ADRP (SEQ ID NO:35),
d) TIP47 (SEQ ID NO:31),
or a functional equivalent thereof.

8. The method of claim 1, wherein said protein of interest is an enzyme.

9. The method of claim 8, wherein said enzyme is an enzyme involved in the biosynthesis of hydrophobic (lipophilic) compounds of interest.

10. The method of claim 9, wherein the enzyme is involved in the biosynthesis of
a) lipophilic vitamins, derivatives and precursors thereof,
b) fatty acids and fatty alcohols, or
c) flavouring substances.

11. The method of claim 1, wherein the bacterial cell is selected from the group consisting of native or recombinant bacteria having the ability to produce inclusion bodies of the PHA-, TAG- or WE-type, TAG-producing nocardioform actinomycetes, TAG-producing Streptomycetes, WE-producing genera Acinetobacter and Alcanivorax, and recombinant strains of the genus Escherichia, Corynebacterium and Bacillus.

12. The method of claim 11, wherein the bacterial cell is Rhodococcus opacus PD630 (DSM 44193) or Mycobacterium smegmatis mc²155 (ATCC 700084).

13. The method of claim 1, wherein said bacterial cell is transformed with an expression construct comprising a coding sequence for said fusion protein under the control of a promoter sequence operable in said bacterial host cell.

14. A method for microbial production of a lipophilic compound of interest, comprising cultivating a recombinant bacterial host comprising intracellular inclusion bodies having at least one enzyme which is involved in the biosynthesis of a lipophilic compound targeted to said inclusion bodies, and cultivating said host under conditions supporting the production of said lipophilic compound.

15. The method of claim 14, wherein the inclusion bodies carrying said lipophilic compound of interest are isolated and said lipophilic compound of interest is recovered from said inclusion bodies.

16. The method of claim 15, wherein said lipophilic compound is
a) lipophilic vitamins, derivatives and precursors thereof,
b) fatty acids and fatty alcohols, or
c) flavouring substances.

17. A fusion protein useful for targeting a protein of interest to an intracellular hydrophobic inclusion body of a bacterial cell, wherein the fusion protein comprises a targeting peptide operatively linked with a protein of interest.

18. The fusion protein of claim 17, which targets the protein of interest to inclusion bodies of the TAG-, WE- or PHA-type.

19. The fusion protein of claim 17, wherein the targeting peptide is a pro- or eukaryotic peptide.

20. The fusion protein of claim 17, wherein the targeting molecule is selected from peptides of bacterial, animal or plant origin.

21. The fusion protein of claim 17, wherein the targeting molecule is
a) derived from a protein associated in its native state with prokaryotic PHA inclusion bodies; or
b) derived from a protein associated in its native state with eukaryotic TAG or WE inclusion bodies.

22. The fusion protein of claim 21, wherein the targeting molecule is selected from poly hydroxyalkanoat body binding phasins, perilipins, Adipose Differentiation Related Proteins (ADRPs)/adipophilins and Tail Interacting Proteins (TIPs).

23. The fusion protein of claim 22, wherein the targeting molecule is selected from:
a) PhaP1 (SEQ ID NO:19),
b) Perilipin A (SEQ ID NO:27),
c) ADRP (SEQ ID NO:35),
d) TIP47 (SEQ ID NO:31),
or a functional equivalent thereof.

24. The fusion protein of claim 17, wherein said protein of interest is an enzyme.

25. The fusion protein of claim 24, wherein said enzyme is an enzyme involved in the biosynthesis of hydrophobic compounds.

26. The fusion protein of claim 25, wherein the enzyme is involved in the biosynthesis of
a) lipophilic vitamins, derivatives and precursors thereof,
b) fatty acids and fatty alcohols, or
c) flavouring substances.

27. A nucleotide sequence encoding the fusion protein of claim 17.

28. An expression vector comprising under the control of at least one regulatory sequence the nucleotide sequence of claim 27.

29. A recombinant bacterial host cell line, carrying the expression vector of claim 28.

30. The recombinant bacterial host cell line of claim 29, wherein the bacterial host cell line is derived from a bacterial cell selected from the group consisting of native or recombinant bacteria having the ability to produce inclusion bodies of the PHA-, TAG- or WE-type, TAG-producing nocardioform actinomycetes, TAG-producing Streptomycetes, WE-producing genera Acinetobacter and Alcanivorax, recombinant strains of the genus Escherichia, Corynebacterium and Bacillus, Rhodococcus opacus PD630 (DSM 44193), and Mycobacterium smegmatis mc²155 (ATCC 700084).
